(19) United States
(12) Patent Application Publication
(10) Pub. No.: US 2025/0057672 A1
(43) Pub. Date: Feb. 20, 2025

(54) COVERED STENT MANUFACTURING METHOD AND COVERED STENT

(71) Applicant: OLYMPUS CORPORATION, Tokyo (JP)

(72) Inventors: Mamoru HASEGAWA, Nagano (JP); Shun NOGUCHI, Tokyo (JP); Yuta SASAKI, Tokyo (JP); Toshiaki HAYASHI, Tokyo (JP)

(21) Appl. No.: 18/935,142

(22) Filed: Nov. 1, 2024

Related U.S. Application Data
(63) Continuation of application No. PCT/JP2022/027996, filed on Jul. 19, 2022.

(51) Int. Cl. A61F 2/90; B29C 65/00; B29K 105/08; B29L 23/00; B32B 1/08; B32B 3/30; B32B 5/02; B32B 7/05; B32B 27/12; B32B 27/32

(52) U.S. Cl. CPC A61F 2/90; B29C 66/63; B29C 66/729; B32B 1/08; B32B 3/30; B32B 5/026; B32B 5/028; B32B 7/05; B32B 27/12; B32B 27/322; A61F 2210/0057; A61F 2210/0076; A61F 2240/001; B29K 2105/0836; B29K 2995/0082; B29L 2023/007; B32B 2307/546; B32B 2535/00

(57) ABSTRACT

Provided is a covered stent manufacturing method including: disposing an inner cover inside a stent body having a mesh structure formed by knitting wires, the stent body having engagement portions, each of which is formed by hooking two bent portions of the wires with each other; disposing an outer cover outside the stent body; forming a slack portion that expands in a radial direction of the stent body in at least one of the covers, the slack portion creating slack that allows movements of the two bent portions in a longitudinal direction and the radial direction of the stent body in at least one of the covers; and joining the covers with each other in regions on an inner side of the mesh of the mesh structure.

### CROSS-REFERENCE TO RELATED APPLICATIONS

This is a continuation of International Application PCT/JP2022/027996 which is hereby incorporated by reference herein in its entirety.

### TECHNICAL FIELD

The present invention relates to a covered stent manufacturing method and a covered stent.

### BACKGROUND ART

In the related art, there is a known covered stent that is disposed in a constricted portion of a lumen in order to release the constriction (for example, see Patent Literatures 1 and 2). A covered stent includes a cover that covers at least one of inside or outside of a tubular stent body. The cover prevents tissue from infiltrating into the interior of the stent placed in a lumen.

In the covered stent of Patent Literature 1, an inner cover and an outer cover are bonded to each other in mesh portions of a mesh-like stent body. Accordingly, the covers are prevented from being twisted when the stent body is expanded, and the inner and outer covers can be bent together with the stent body.

In the covered stent of Patent Literature 2, a plurality of bonding portions in which an inner cover and an outer cover are bonded to each other are formed with spacings between each other and pockets that allow a stent body to move are formed between the bonding portions adjacent to each other.

Accordingly, the covered stent can be compressed in a radial direction with a small force.

### CITATION LIST

### Patent Literature

{PTL 1}Japanese Unexamined Patent Application, Publication No. 2005-52419
{PTL 2}Publication of Japanese Patent No. 4592953

### SUMMARY OF INVENTION

An aspect of the present invention is a covered stent manufacturing method including: disposing an inner cover inside a stent body having a mesh structure formed by knitting wires, the stent body having engagement portions, each of which is formed by hooking two bent portions of the wires with each other; disposing an outer cover outside the stent body; forming a slack portion that expands in a radial direction of the stent body in at least one of the inner cover or the outer cover, the slack portion creating slack that allows movements of the two bent portions in a longitudinal direction and the radial direction of the stent body in at least one of the inner cover or the outer cover; and joining the inner cover and the outer cover with each other in regions on an inner side of the mesh of the mesh structure.

Another aspect of the present invention is a covered stent including: a stent body that has a mesh structure formed by knitting wires and that has engagement portions, each of which is formed by hooking two bent portions of the wires with each other; an inner cover that covers an inside of the stent body; and an outer cover that covers an outside of the stent body, wherein at least one of the inner cover or the outer cover has a slack portion that expands in a radial direction of the stent body, the slack portion creating slack that allows movements of the two bent portions in a longitudinal direction and the radial direction of the stent body in at least one of the inner cover or the outer cover, and the inner cover and the outer cover are joined with each other in regions on an inner side of the mesh of the mesh structure.

Another aspect of the present invention is a covered stent manufacturing method including: disposing, at least inside or outside a stent body, a cover made of an ePTFE in an orientation in which a stretching direction of the ePTFE is aligned with a stretching direction of the stent body when the stent body is contracted in a radial direction; partially connecting the cover with the stent body; and creating slack in the cover by contracting the stent body in the radial direction and stretching the cover.

Another aspect of the present invention is a covered stent including: a stent body; and a cover that is made of an ePTFE, that covers at least one of inside or outside of the stent body, and that is partially connected to the stent body, wherein the cover is disposed in an orientation in which a stretching direction of the ePTFE is aligned with a stretching direction of the stent body when the stent body is contracted in a radial direction, and the cover has slack in a longitudinal direction of the stent body in a state in which the stent body is expanded in the radial direction.

### BRIEF DESCRIPTION OF DRAWINGS

FIG. 1 is a side view of a covered stent according to a first embodiment of the present invention.

FIG. 2A is a partially enlarged plan view of a stent body.

FIG. 2B is a sectional view of the covered stent in FIG. 2A taken along the line I-I.

FIG. 3 is a diagram for explaining the shapes of the stent body, an inner cover, and an outer cover of the covered stent in a straight shape.

FIG. 4 is a diagram for explaining the shapes of the stent body, the inner cover, and the outer cover of the covered stent in a bent shape.

FIG. 5 is a flowchart of a covered stent manufacturing method according to the first embodiment of the present invention.

FIG. 6 is a diagram showing a core rod used in the manufacturing method in FIG. 5.

FIG. 7 is a diagram for explaining steps SA1 to SA5 of the manufacturing method in FIG. 5.

FIG. 8A is a diagram for showing a modification of depressions.

FIG. 8B is a sectional view of the covered stent showing a state in which the depressions in FIG. 8A are compressed.

FIG. 9A is a diagram for showing another modification of the depressions.

FIG. 9B is a sectional view of the covered stent showing a state in which the depressions in FIG. 9A are compressed.

FIG. 10 is a flowchart of a covered stent manufacturing method according to a second embodiment of the present invention.

FIG. 11 is a diagram for explaining steps SB1 to SB5 of the manufacturing method in FIG. 10.

FIG. 12A is a partially enlarged plan view of the stent body showing an example of joining portions.

FIG. 12B is a partially enlarged plan view of the stent body showing another example of the joining portions.

FIG. 13 is a flowchart of a first modification of the manufacturing method in FIG. 10.

FIG. 14 is a diagram for explaining steps SB1 to SB5 of the manufacturing method in FIG. 13.

FIG. 15 is a flowchart of a second modification of the manufacturing method in FIG. 10.

FIG. 16 is a diagram for explaining step SB45 of the manufacturing method in FIG. 15.

FIG. 17 is a flowchart of a third modification of the manufacturing method in FIG. 10.

FIG. 18 is a diagram for explaining steps SB1 to SB5 of the manufacturing method in FIG. 17.

FIG. 19 is a flowchart of a covered stent manufacturing method according to a third embodiment of the present invention.

FIG. 20 is a diagram showing a core rod used in the manufacturing method in FIG. 19.

FIG. 21 is a diagram for explaining steps SC1 to SC4 of the manufacturing method in FIG. 19.

FIG. 22 is a flowchart of a covered stent manufacturing method according to a fourth embodiment of the present invention.

FIG. 23A is a diagram showing a core rod and a jig used in the manufacturing method in FIG. 22.

FIG. 23B is a diagram showing an example of the jig in FIG. 23A constituted of two half bodies.

FIG. 23C is a diagram showing another example of the jig in FIG. 23A constituted of two half bodies.

FIG. 24 is a diagram for explaining steps SD1 to SD6 of the manufacturing method in FIG. 22.

FIG. 25A is a perspective view of a jig used in a covered stent manufacturing method according to a fifth embodiment of the present invention.

FIG. 25B is a perspective view of a modification of the jig in FIG. 25A.

FIG. 26A is a diagram for explaining step SD5.

FIG. 26B is a diagram for explaining step SD5.

FIG. 27A is a diagram showing another modification of the depressions.

FIG. 27B is a diagram showing another modification of the depressions.

FIG. 28A is a side view of a covered stent according to a sixth embodiment of the present invention.

FIG. 28B is a side view showing the covered stent in FIG. 28A in a contracted state.

FIG. 29 is a flowchart of a covered stent manufacturing method according to the sixth embodiment of the present invention.

FIG. 30 is a diagram for explaining step SE1 of the manufacturing method in FIG. 29.

FIG. 31 is a diagram for explaining steps SE2 to SE6 of the manufacturing method in FIG. 29.

FIG. 32 is a side view of a covered stent according to a seventh embodiment of the present invention.

FIG. 33 is a flowchart of a covered stent manufacturing method according to the seventh embodiment of the present invention.

FIG. 34 is a diagram for explaining steps SF2 to SF6 of the manufacturing method in FIG. 33.

FIG. 35 is a side view of a modification of the covered stents of the sixth and seventh embodiments.

### DESCRIPTION OF EMBODIMENTS

### First Embodiment

A covered stent manufacturing method and a covered stent according to a first embodiment of the present invention will be described with reference to the drawings.

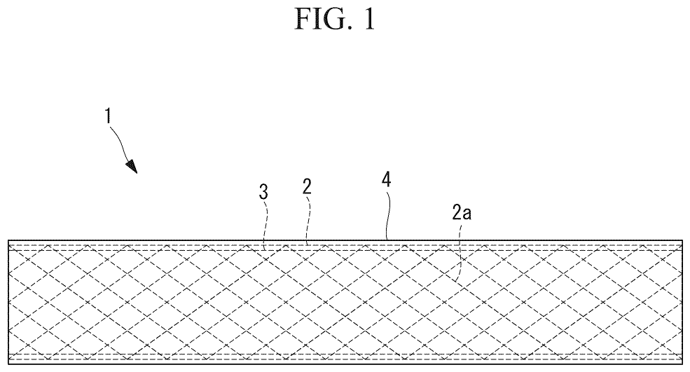

As shown in FIG. 1, a covered stent 1 according to this embodiment includes a tubular stent body 2, a tubular inner cover 3 that covers the inside of the stent body 2, and a tubular outer cover 4 that covers the outside of the stent body 2.

The stent body 2 is formed by knitting one or more wires 2a while the wires 2a are bent in a zigzag manner and wound about a center axis and has a mesh structure in which numerous rhombus meshes are arrayed in a circumferential direction and a longitudinal direction. The stent body 2 can be contracted in a radial direction. The covered stent 1 is mounted on a delivery system in a contracted state, is transported into a body cavity by means of the delivery system, and is expanded in the radial direction in the body cavity.

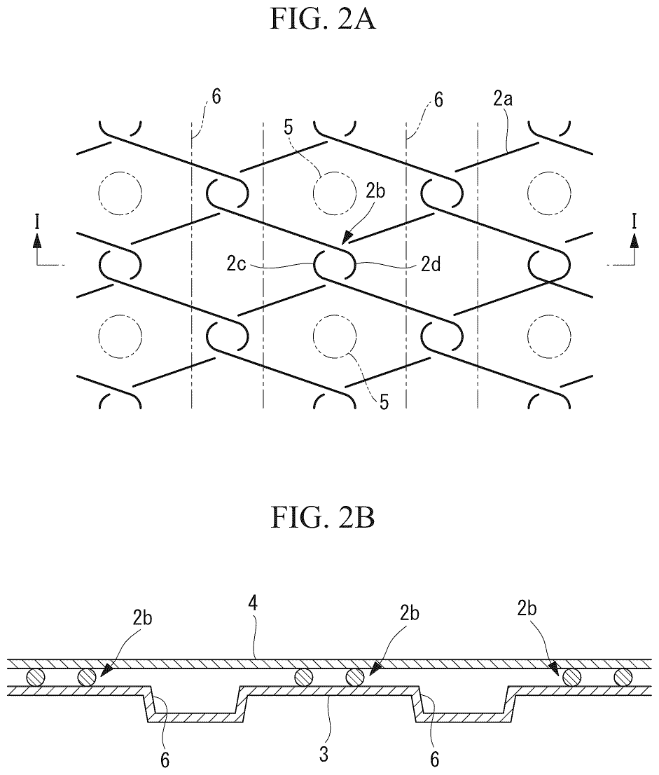

As shown in FIG. 2A, the stent body 2 has engagement portions 2b, each of which consists of two bent portions 2c, 2d of the wires 2a hooked on each other in the longitudinal direction of the stent body 2. The bent portions 2c are peak portions that are bent toward one end of the stent body 2 and protrude toward the other end thereof. The bent portions 2d are valley portions that are bent toward the other end of the stent body 2 and protrude toward the one end thereof. The zigzagging wires 2a have the peak portions 2c and the valley portions 2d that are arrayed in the circumferential direction in an alternating manner. The engagement portions 2b are formed by hooking the peak portions 2c in one column with the valley portions 2d in another column adjacent thereto.

Accordingly, the two bent portions 2c, 2d are linked with each other so as to be freely displaced in the longitudinal direction and the radial direction. As a result of the three-dimensional displacement of the bent portions 2c, 2d in the engagement portions 2b, the stent body 2 can be easily bent by generating nearly or entirely no axial force.

The inner cover 3 and the outer cover 4 are sheets made of an expanded polytetrafluoroethylene (ePTFE). The material of the covers 3, 4 may be other materials that have biocompatibility and flexibility and that are generally used in stents, for example, silicone. The inner cover 3 and the outer cover 4 are joined with each other in joining portions 5, which are portions of regions on an inner side of the mesh, and are separated from each other in portions other than the joining portions 5.

As shown in FIG. 2B, the inner cover 3 has a plurality of slack portions 6 that are arrayed in the longitudinal direction with spacings therebetween. Each of the slack portions 6 consists of a depression that extends over the entire circumference of the inner cover 3 and expands radially inward, and is, for example, a U-shaped groove having a rectangular cross-sectional shape in a longitudinal cross section in the longitudinal direction. Some of the engagement portions 2b are disposed between the slack portions 6 and the outer cover 4.

The slack portions 6 form spaces between the inner cover 3 and the outer cover 4 and, in addition, create slack in the inner cover 3. The inner cover 3 having slack can freely deform in accordance with an external force and allows movements of the pair of bent portions 2c, 2d, forming each of the engagement portions 2b, in the longitudinal direction and the radial direction. Therefore, the movable ranges of the bent portions 2c, 2d in the engagement portions 2b increase and the covers 3, 4 are prevented from hindering the three-dimensional displacements of the bent portions 2c, 2d when the covered stent 1 is bent. Accordingly, a low axial force and high flexibility are realized in the covered stent 1.

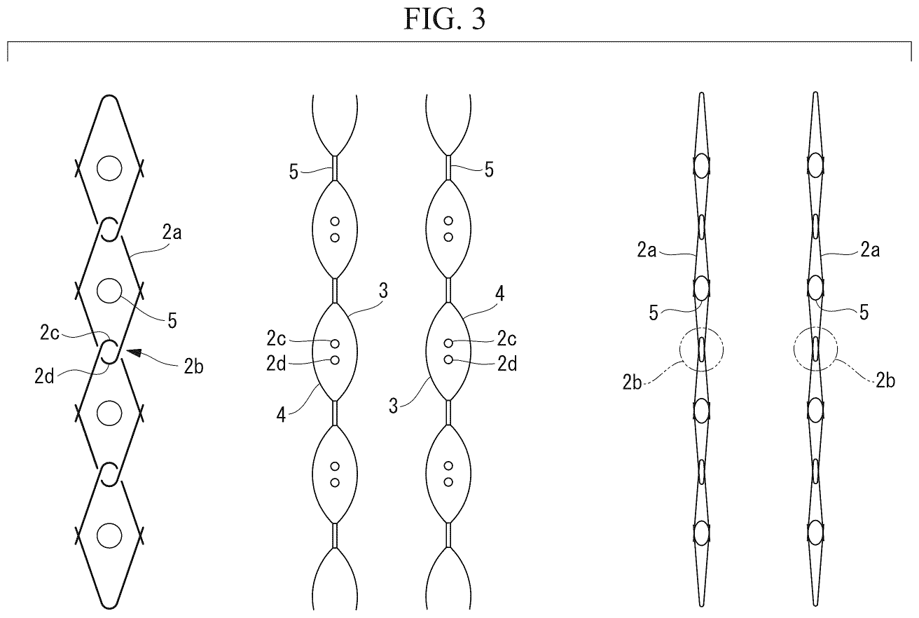

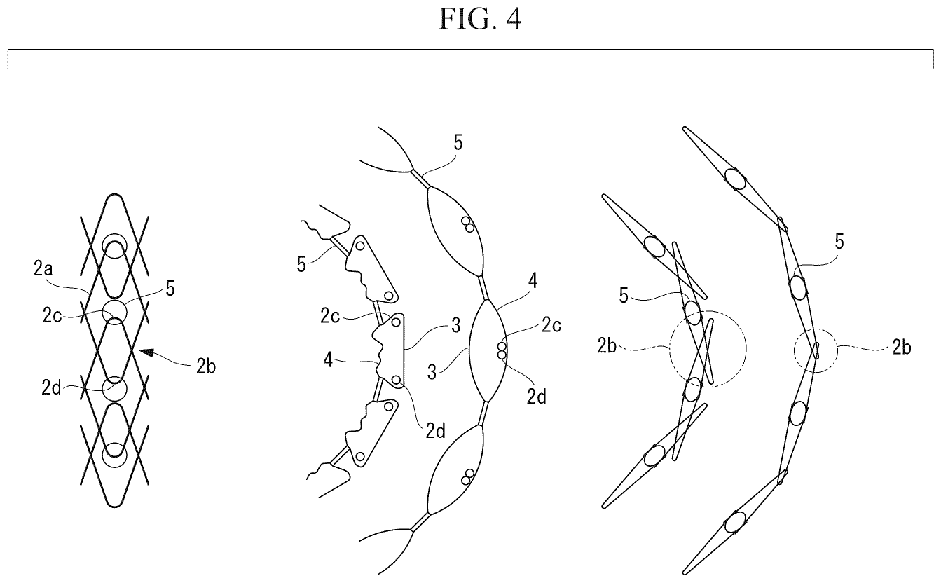

FIGS. 3 and 4 describe the relationship between the displacement of the bent portions 2c, 2d due to bending of the covered stent 1 and the deformation of the covers 3, 4 having slack. In FIGS. 3 and 4, the left diagram is a diagram in which the stent body 2 inside a bend is viewed from the front, the center diagram is a schematic diagram of the cover 3 inside the bend and the cover 4 outside the bend, and the right diagram is a schematic diagram of the wires 2a inside and outside the bend. In FIGS. 3 and 4, the inner cover 3 and the outer cover 4 both have slack.

When the covered stent 1 is bent from the straight shape in FIG. 3 to the bent shape in FIG. 4, apexes of the two bent portions 2c, 2d in each of the engagement portions 2b are displaced in opposite directions with respect to each other in the longitudinal direction inside and outside the bend and displaced radially inward. At this time, the covers 3, 4 having slack are deformed in accordance with the longitudinal direction and radially inward displacements of the bent portions 2c, 2d. Therefore, when the covered stent 1 is bent, the two bent portions 2c, 2d can be displaced in the same manner as in the case in which the covers 3, 4 are not present, and thus, it is possible to realize a low axial force.

Next, the covered stent manufacturing method according to this embodiment will be described.

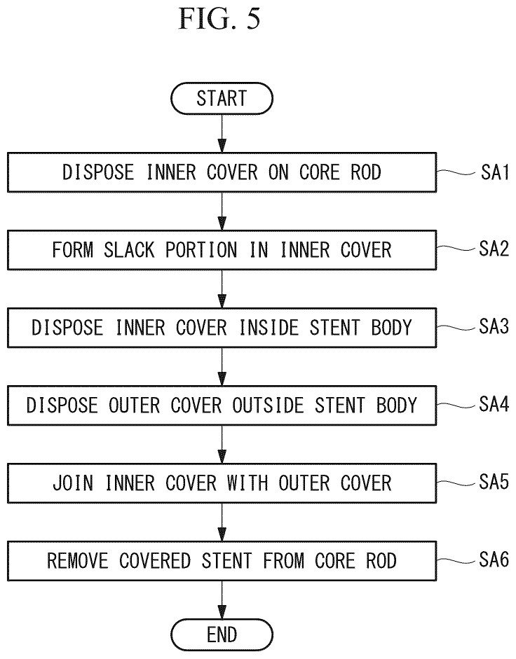

As shown in FIG. 5, the covered stent manufacturing method includes: step SA1 of disposing the inner cover 3 on a first jig 20; step SA2 of forming the slack portions 6 in the inner cover 3; step SA3 of disposing the inner cover 3 inside the stent body 2; step SA4 of disposing the outer cover 4 outside the stent body 2; step SA5 of joining the inner cover 3 and the outer cover 4 with each other; and SA6 of removing the covered stent 1 from the jig 20.

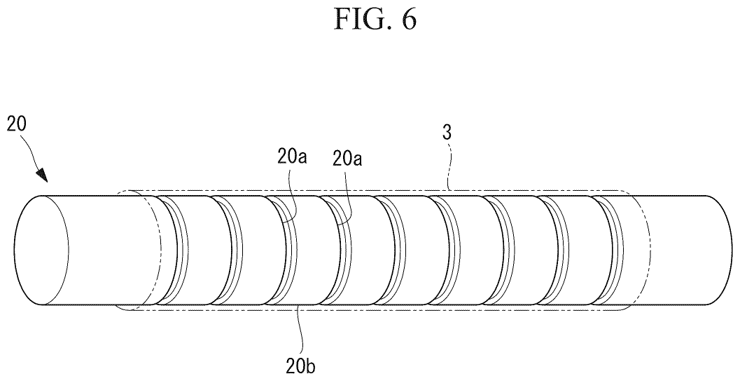

As shown in FIG. 6, the jig 20 is a columnar core rod and, on an outer surface of the core rod 20, a plurality of depressions 20a that are arrayed in the longitudinal direction with spacings therebetween are formed. The plurality of depressions 20a are structures for forming the slack portions 6. Each of the depressions 20a extends over the entire circumference and is depressed radially inward. Protrusions 20b are formed between the depressions 20a adjacent to each other.

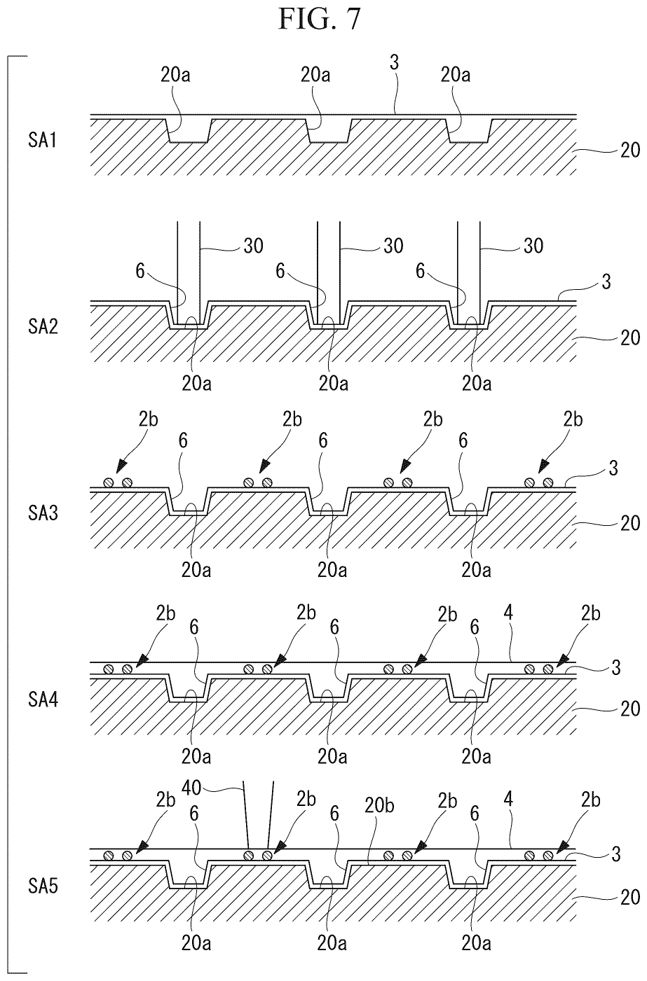

As shown in FIG. 7, the inner cover 3 is disposed on the outer surface of the core rod 20 (step SA1). The form of the inner cover 3 is selected from a tube, a tape, and a sheet. In the case of a tape shape or a sheet shape, the inner cover 3 is disposed on the core rod 20 after being molded and joined into a tube shape in advance or is wound around the core rod 20 without any gap.

Next, the slack portions 6 are formed by pushing the inner cover 3 into the depressions 20a by using push tools 30 (step SA2). The push tools 30 are, for example, rod-shaped or ring-shaped members. As a result of the inner cover 3 being deformed along inner surfaces of the depressions 20a, the slack portions 6 having dimensions and shapes corresponding to the dimensions and the shapes of the depressions 20a are formed. In order to reliably cause the inner cover 3 to be deformed along the inner surfaces of the depressions 20a, a distal-end portion of each of the push tools 30 may have an outer surface shape that is complementary to the inner surface shape of each of the depressions 20a.

Next, the stent body 2 is disposed on an outer surface of the inner cover 3 by inserting the core rod 20 into the stent body 2 (step SA3). The stent body 2 and the inner cover 3 are aligned with respect to each other at positions at which the engagement portions 2b are disposed at the slack portions 6.

Next, the outer cover 4 is disposed on the stent body 2 and the stent body 2 is covered with the outer cover 4 (step SA4). As in the inner cover 3, the form of the outer cover 4 is selected from a tube, a tape, and a sheet. In the case of a tube shape, the core rod 20 on which the inner cover 3 and the stent body 2 are disposed is inserted into the outer cover 4. In the case of a tape shape or a sheet shape, the core rod 20 is inserted into the outer cover 4 molded into a tube shape in advance or the outer cover 4 is wound around the core rod 20 over the entire circumference thereof.

Next, in some regions on an inner side of the mesh of the stent body 2, the inner cover 3 and the outer cover 4 are joined by using a joining tool 40 by means of a method such as thermocompression bonding, and the joining portions 5 are consequently formed (step SAS). The regions to be joined are regions on the inner side of the mesh on the protrusions 20b, and each of the joining portions 5 is formed between the two engagement portions 2b disposed at the two slack portions 6 adjacent to each other (see FIG. 2A).

Next, by removing the core rod 20 from inside the inner cover 3, the covered stent 1 having the slack portions 6 is manufactured (step SA6).

Accordingly, with the manufacturing method of this embodiment, it is possible to manufacture the covered stent 1 with a low axial force and high flexibility, in which the inner cover 3 has slack due to the slack portions 6 and the covers 3, 4 do not hinder bending of the stent body 2.

In addition, the movable ranges of the bent portions 2c, 2d depend on the dimensions of the slack portions 6. In step SA2, the slack portions 6 having the dimensions equivalent to those of the depressions 20a are formed by pushing the inner cover 3 into the depressions 20a of the core rod 20.

Accordingly, it is possible to form the slack portions 6 having desired dimensions by easily and accurately controlling the dimensions of the slack portions 6, such as the depths and the widths thereof, and it is possible to reliably achieve desired bending properties in the covered stent 1. In addition, by varying the dimensions, the shapes, etc. of the depressions 20a at different positions, it is possible to easily form the slack portions 6 having different sizes and shapes at different positions.

Excessive slack in the inner cover 3 causes the diameter of the covered stent 1 in the contracted state to increase, and, consequently, a sliding resistance of the covered stent 1 with respect to the delivery system increases when the covered stent 1 is released from the delivery system, and thus, a large operating force is required. In addition, excessive slack in the inner cover 3 could decrease the volume of a hollow portion inside the covered stent 1 through which a substance passes in the body cavity. Therefore, it is important to control the dimensions of the slack portions 6 to be desired dimensions. With this embodiment, as described above, as a result of using the core rod 20 having the depressions 20a, it is possible to easily and accurately control the dimensions of the slack portions 6.

In addition, of the covers 3, 4 forming a double structure, only the inner cover 3 has the slack portions 6 and the outer cover 4 does not have the slack portions. Therefore, it is possible to decrease the sliding resistance exerted when the covered stent 1 is released from the delivery system.

In this embodiment, the slack portions 6 are U-shaped grooves having flat bottom walls; however, the shapes of the slack portions 6 are not limited thereto and can be changed, as appropriate.

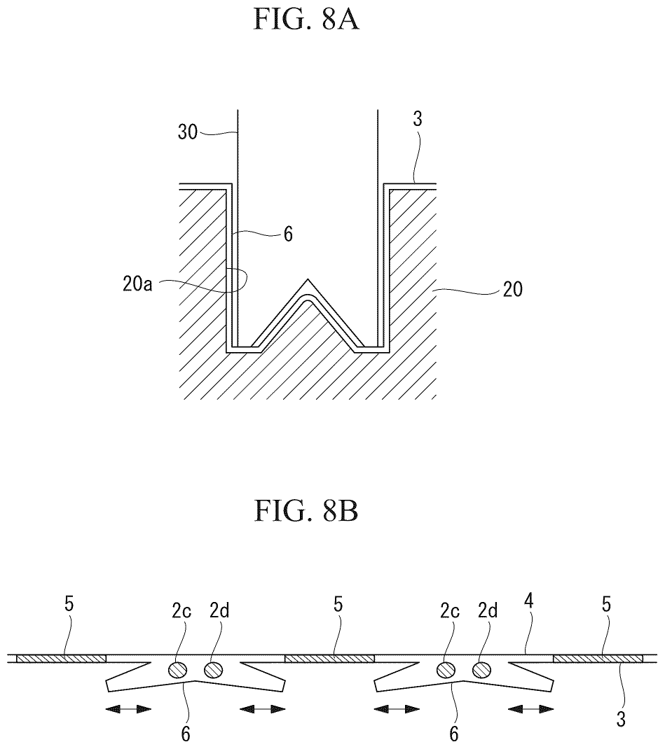

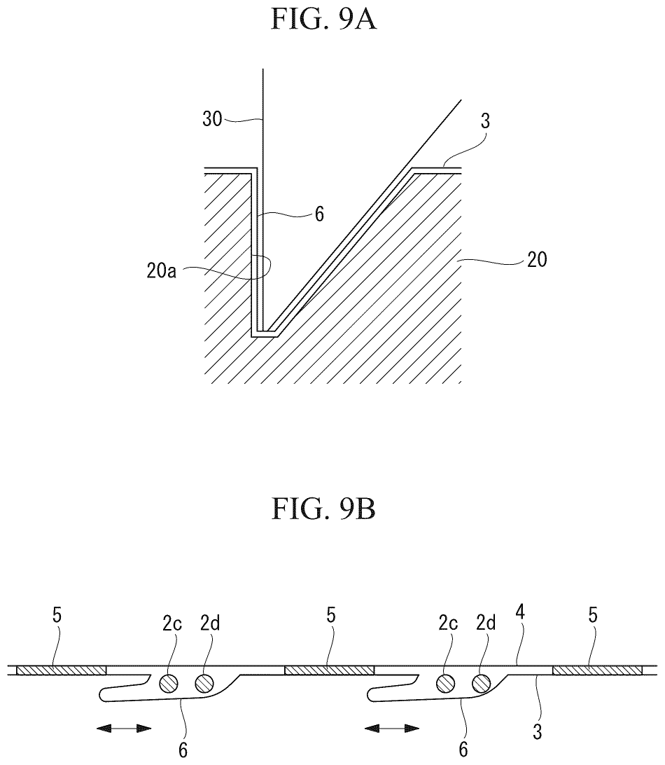

FIGS. 8A and 9A show other examples of the longitudinal cross-sectional shape of the slack portions 6. A slack portion 6 in FIG. 8A has a substantially M-shape and has two side walls that are parallel to each other in the radial direction and a bent bottom wall that protrudes radially outward. A slack portion 6 in FIG. 9A has a substantially V-shape having two side walls that form an angle between each other, wherein one of the side walls is parallel to the radial direction and the other side wall is inclined with respect to the radial direction. Such slack portions 6 are formed by pushing the inner cover 3 into substantially M-shaped or substantially V-shaped depressions 20a. The distal-end portion of each of the push tools 30 may have a shape corresponding to the shape of each of the depressions 20a.

FIGS. 8B and 9B show the slack portions 6 folded in the radial direction with a compression force.

In the case of the substantially M-shaped slack portions 6, the two side walls are folded outward and stacked on portions of the inner cover 3 constituting the slack portions 6 in the radial direction outside openings of the slack portions 6. In the case of the substantially V-shaped slack portions 6, the two side walls are folded outward in the same direction and stacked on the portions of the inner cover 3 constituting the slack portions 6 in the radial direction outside openings of the slack portions 6.

As described above, the substantially M-shaped and substantially V-shaped slack portions 6 are deformed into the prescribed folded shapes due to a compression force in the radial direction. Therefore, when the covered stent 1 is mounted on the delivery system, it is possible to fold the slack portions 6 into the prescribed shapes simply by compressing the covered stent 1 in the radial direction.

The thickness increases in the portions in which the portions of the inner cover 3 are stacked in the radial direction due to folding of the slack portions 6 (see the areas indicated by arrows in FIGS. 8B and 9B). Such thick portions are preferably disposed at positions displaced from the engagement portions 2b in which the two portions 2c, 2d of the wires 2a are stacked in the radial direction. With the slack portions 6 in FIGS. 8A to 9B, it is possible to control the folded shapes of the slack portions 6 so that the thick portions are disposed in regions in which interference with the engagement portions 2b does not occur.

### Second Embodiment

Next, a covered stent manufacturing method and a covered stent according to a second embodiment of the present invention will be described.

In this embodiment, configurations that are different from those of the first embodiment will be described and configurations that are the same as those of the first embodiment will be given the same reference signs, and the descriptions thereof will be omitted.

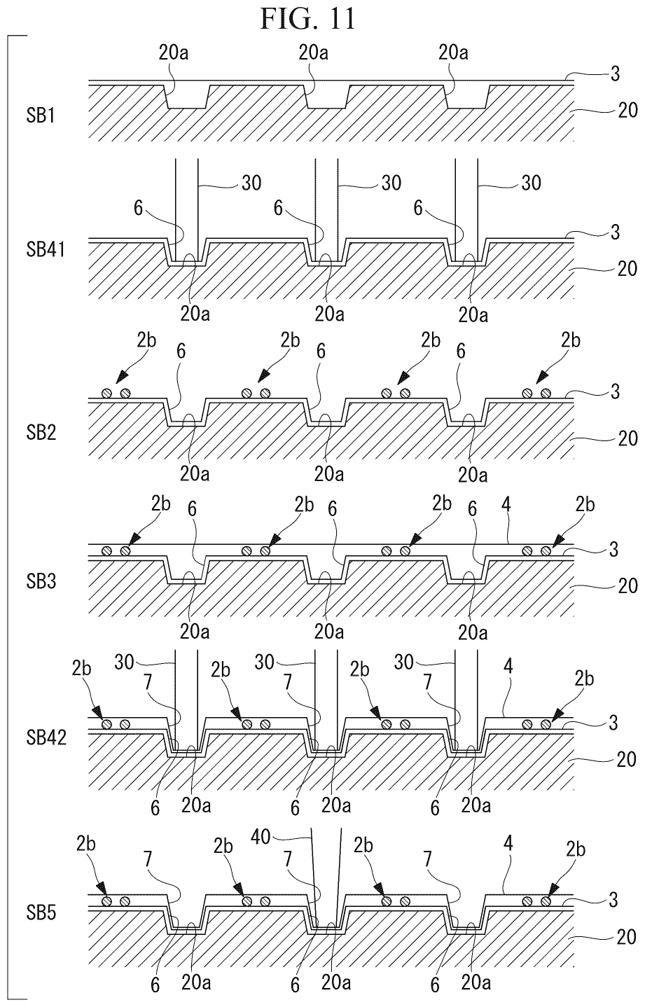

The covered stent according to this embodiment includes the stent body 2, the inner cover 3, and the outer cover 4. The covered stent of this embodiment differs from the covered stent 1 of the first embodiment in that the inner cover 3 has slack portions 6 and the outer cover 4 has slack portions 7 (see FIG. 11).

The outer cover 4 has the plurality of slack portions 7 that are arrayed in the longitudinal direction of the outer cover 4 with spacings therebetween and that are formed at the same positions as the slack portions 6. As in the slack portions 6, each of the slack portions 7 consists of a depression that extends over the entire circumference of the outer cover 4 and expands radially inward, and is, for example, a U-shaped groove having a rectangular cross-sectional shape in a longitudinal cross section in the longitudinal direction. Some of the engagement portions 2b are disposed between the slack portions 6 and the slack portions 7.

As in the inner cover 3, the outer cover 4 in which slack is created by the slack portions 7 can freely deform in accordance with an external force and allows movements of the pair of bent portions 2c, 2d, forming each of the engagement portions 2b, in the longitudinal direction and the radial direction. Therefore, the covers 3, 4 are prevented from hindering the three-dimensional displacements of the bent portions 2c, 2d when the covered stent is bent. Accordingly, a low axial force and high flexibility are realized in the covered stent.

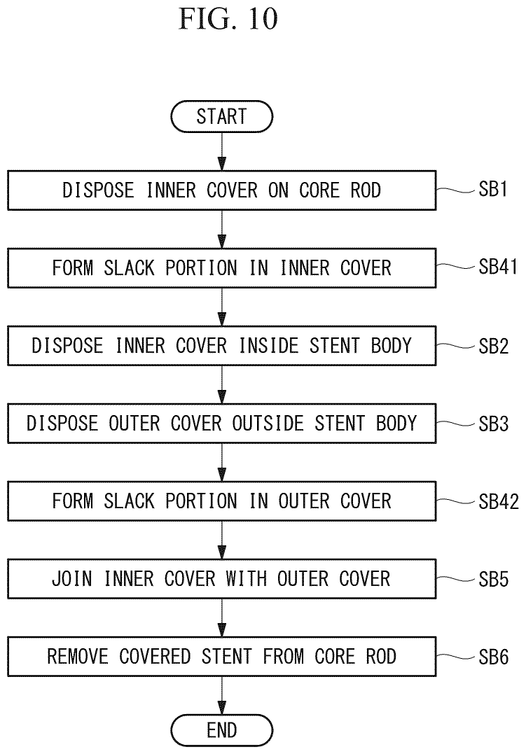

As shown in FIG. 10, the covered stent manufacturing method according to this embodiment includes: step SB1 of disposing the inner cover 3 on the jig 20; step SB2 of disposing the inner cover 3 inside the stent body 2; step SB3 of disposing the outer cover 4 outside the stent body 2; step SB41 of forming the slack portions 6 in the inner cover 3; step SB42 of forming the slack portions 7 in the outer cover 4; step SB5 of joining the inner cover 3 and the outer cover 4 with each other; and step SB6 of removing the covered stent from the jig 20.

The steps of forming the slack portions 6 and 7 include: step SB41 of forming the slack portions 6 in the inner cover 3 before step SB3; and step SB42 of forming the slack portions 7 in the outer cover 4 after step SB3.

As shown in FIG. 11, as in step SA1, the inner cover 3 is disposed on the outer surface of the core rod 20 (step SB1).

Next, as in step SA2, the slack portions 6 are formed by pushing the inner cover 3 into the depressions 20a by using the push tools 30 (step SB41).

Next, as in step SA3, the stent body 2 is disposed on the outer surface of the inner cover 3 by inserting the core rod 20 into the stent body 2 (step SB2).

Next, as in step SA4, the outer cover 4 is disposed on the stent body 2 and the stent body 2 is covered with the outer cover 4 (step SB3).

Next, the slack portions 7 are formed by pushing the outer cover 4 into the depressions 20a by using the push tools 30 (step SB42).

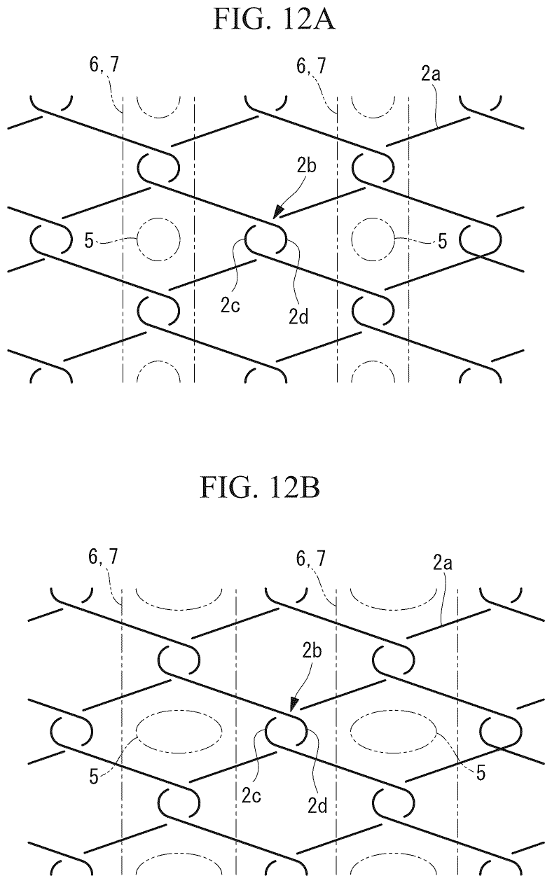

Next, in some regions on the inner side of the mesh of the stent body 2, the inner cover 3 and the outer cover 4 are joined by using the joining tool 40 by means of a method such as thermocompression bonding, and the joining portions 5 are consequently formed (step SB5). As shown in FIG. 12A, the regions to be joined are regions between the two engagement portions 2b adjacent to each other disposed in the same slack portions 6, 7, and the joining portions 5 are formed in the slack portions 6, 7. The regions to be joined may be the same as the joining portions 5 in the first embodiment. In addition, as shown in FIG. 12B, joining portions 5 having large areas may be formed in wide slack portions 6, 7.

Next, by removing the core rod 20 from inside the inner cover 3, the covered stent having the slack portions 6 and 7 is manufactured (step SB6).

Accordingly, with the manufacturing method of this embodiment, it is possible to manufacture the covered stent with a low axial force and high flexibility, in which the inner covers 3, 4 both have slack due to the slack portions 6, 7 and the covers 3, 4 do not hinder bending of the stent body 2.

In addition, the slack portions 6, 7 having the dimensions equivalent to those of the depressions 20a are formed by pushing the inner covers 3, 4 into the depressions 20a of the core rod 20. Accordingly, it is possible to form the slack portions 6, 7 having desired dimensions by easily and accurately controlling the dimensions of the slack portions 6, 7.

In this embodiment, the slack portions 6, 7 may have shapes that deform into prescribed folded shapes, as shown in FIGS. 8A and 9A, as in the first embodiment.

In this embodiment, the slack portions 6 and the slack portions 7 are formed in different steps SB41 and SB42; however, alternatively, the slack portions 6 and 7 may be simultaneously formed in single step SB4.

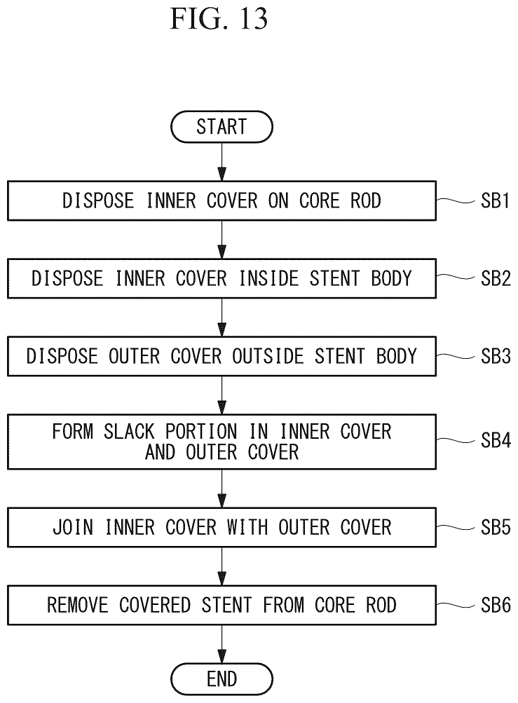

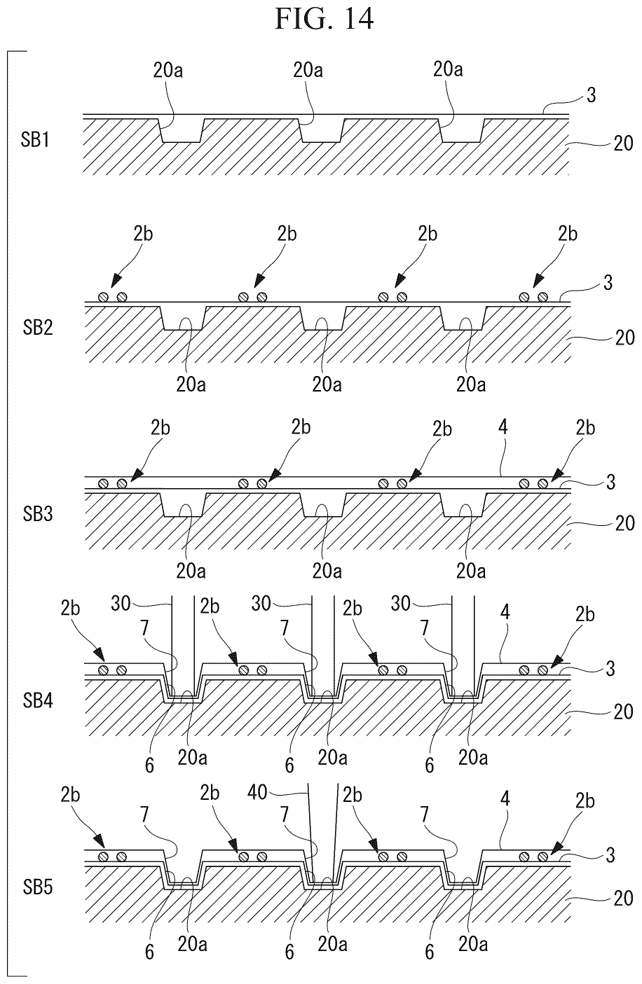

FIGS. 13 and 14 describe a first modification of the manufacturing method of the second embodiment. In this modification, step SB4 of forming the slack portions 6 and 7 is performed by simultaneously pushing the inner cover 3 and the outer cover 4 into the depressions 20a by using the push tools 30 after step SB3. With this modification, it is possible to decrease the number of steps.

The formation of the slack portions 6 and 7 and the joining of the covers 3, 4 may simultaneously be performed in the single step SB45.

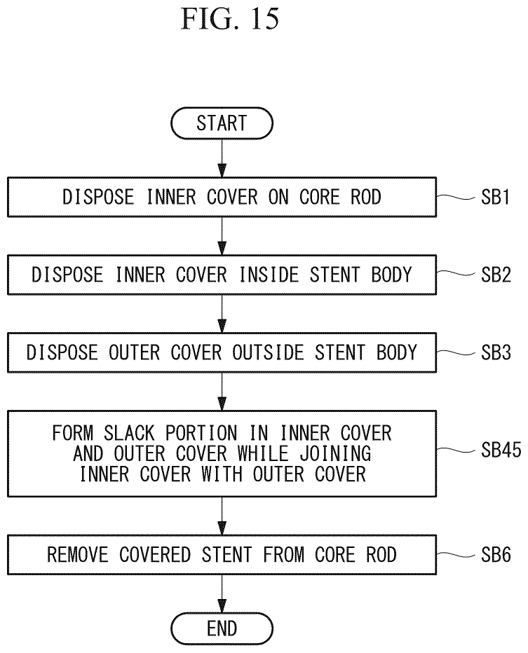

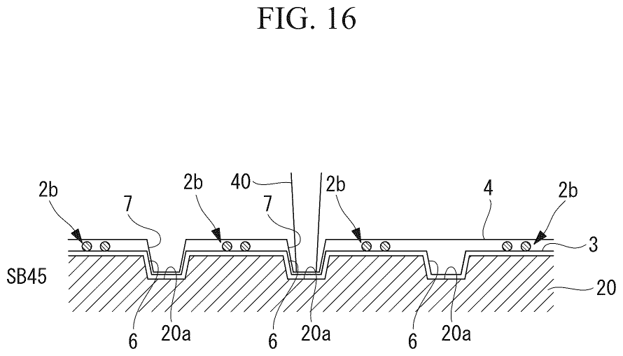

FIGS. 15 and 16 describe a second modification of the manufacturing method of the second embodiment. In this modification, step SB45 is performed by joining the covers 3, 4 with each other by means of a joining tool 40, such as a pressurizing or heating pin, while the covers 3, 4 are simultaneously being pushed into the depressions 20a by using the joining tool 40, after step SB3. With this modification, it is possible to further decrease the number of steps.

In this embodiment, the slack portions 6 are formed and the covers 3, 4 are subsequently joined with each other; however, alternatively, the covers 3, 4 may be joined with each other (step SB5) and the slack portions 6, 7 may subsequently be formed (step SB4).

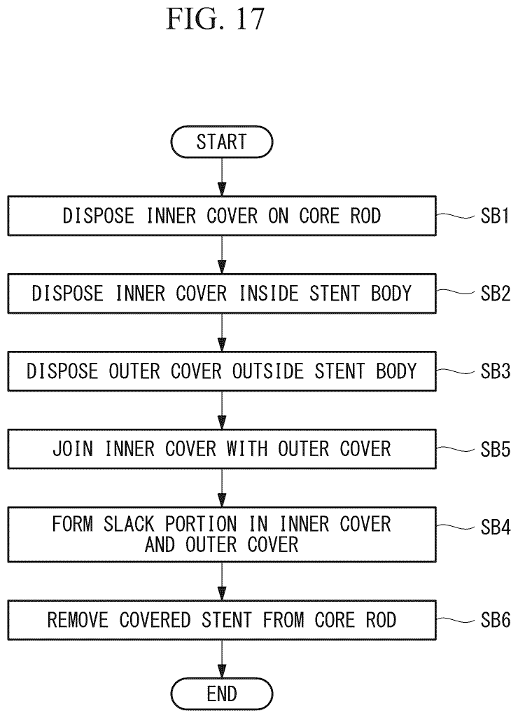

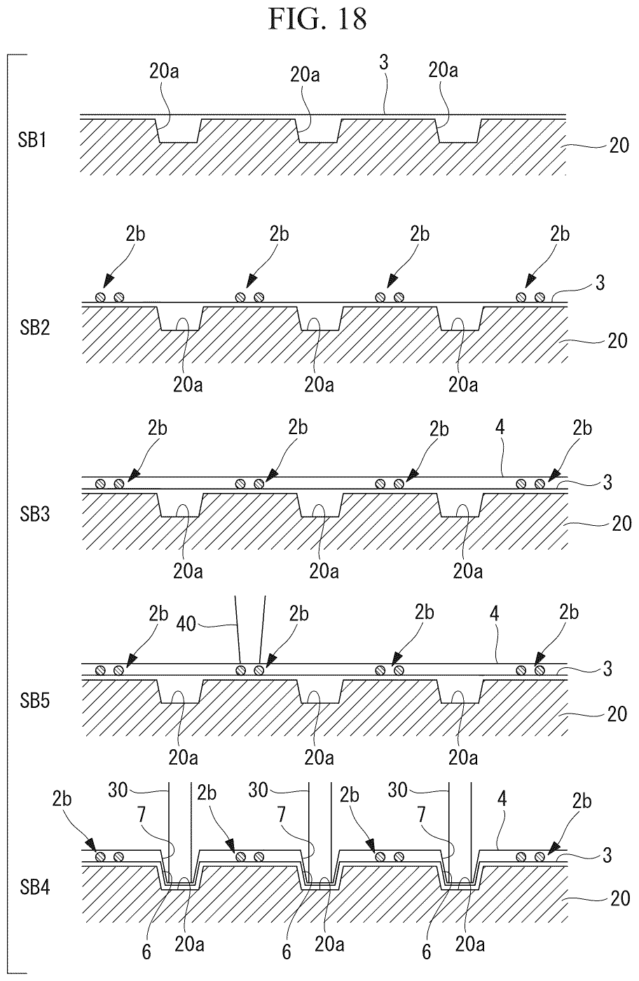

FIGS. 17 and 18 describe a third modification of the manufacturing method of the second embodiment. In this modification, the inner cover 3 and the outer cover 4 are joined with each other in the regions on the inner side of the mesh on the protrusions 20b and the joining portions 5 are formed (step SB5), as in the joining portions 5 in the first embodiment (see FIG. 2A). Subsequently, the covers 3, 4 are pushed into the depressions 20a, and the slack portions 6, 7 are formed as a result of the stretching of the covers 3, 4 (step SB4).

### Third Embodiment

Next, a covered stent manufacturing method and a covered stent according to a third embodiment of the present invention will be described.

In this embodiment, configurations that are different from those of the first embodiment will be described and configurations that are the same as those of the first embodiment will be given the same reference signs, and the descriptions thereof will be omitted.

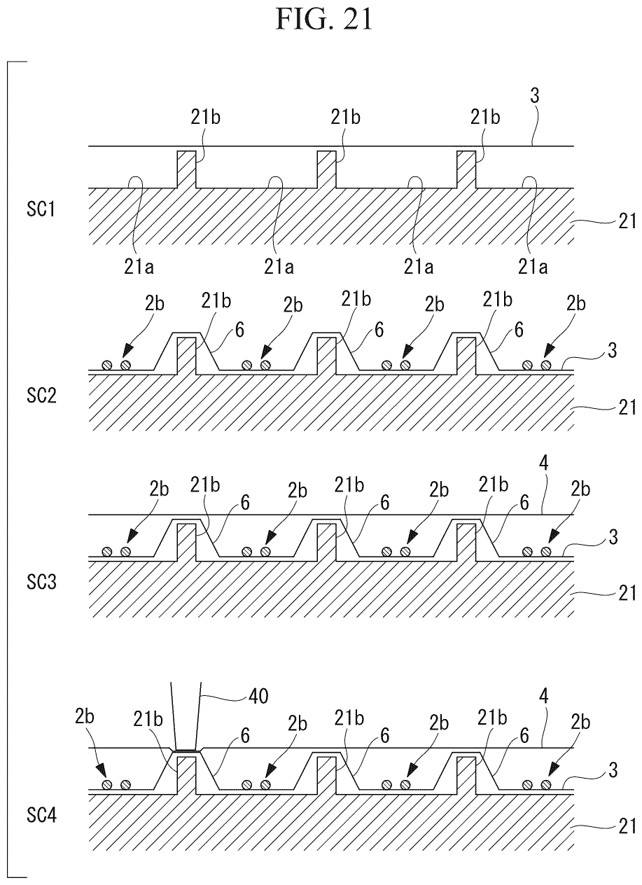

The covered stent according to this embodiment includes the stent body 2, the inner cover 3, and the outer cover 4. The covered stent of this embodiment differs from the covered stent 1 of the first embodiment in that the stent body 2 is disposed at the slack portions 6 of the inner cover 3 (see FIG. 21).

The inner cover 3 has, in addition to the plurality of slack portions 6 that extend in the circumferential direction and that are arrayed in the longitudinal direction, a plurality of slack portions 6 that extend in the longitudinal direction and that are arrayed in the circumferential direction. the slack portions 6 are continuous with each other and form, as a whole, grid-like depressions in the inner cover 3. The wires 2a of the stent body 2 are disposed inside the slack portions 6.

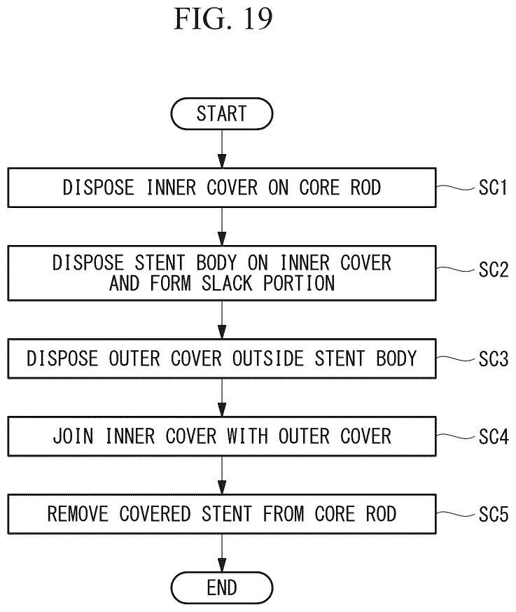

As shown in FIG. 19, the covered stent manufacturing method according to this embodiment includes: step SC1 of disposing the inner cover 3 on a first jig 21; step SC2 of forming the slack portions 6 by disposing the stent body 2 outside the inner cover 3; step SC3 of disposing the outer cover 4 outside the stent body 2; step SC4 of joining the inner cover 3 and the outer cover 4 with each other; and step SC5 of removing the covered stent from the jig 21.

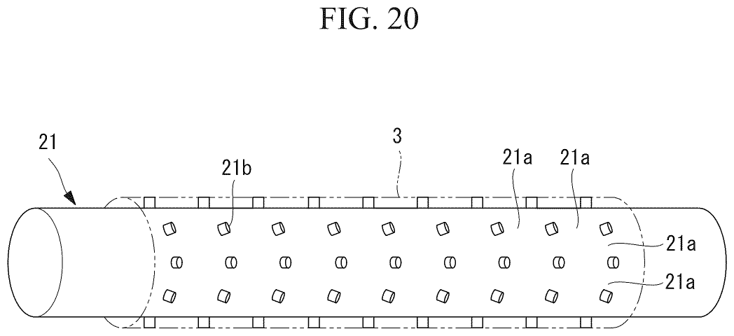

As shown in FIG. 20, the jig 21 has a plurality of protrusions 21b that are arrayed in the longitudinal direction and the circumferential direction with spacings therebetween. The protrusions 21b are columnar protrusions that protrude radially outward from an outer circumferential surface of the jig 21. Between the protrusions 21b, depressions 21a that extend in the circumferential direction and depressions 21a that extend in the longitudinal direction are formed, and the depressions 21a are continuous with each other.

As shown in FIG. 21, the inner cover 3 is disposed on the protrusions 21b of the core rod 20 (step SC1).

Next, the stent body 2 is disposed on the outer surface of the inner cover 3 so that the protrusions 21b are positioned on the inner side of the mesh. Accordingly, the inner cover 3 is depressed radially inward in the regions of the depressions 21a and the slack portions 6 are formed in the depressions 21a (step SC2).

Next, the outer cover 4 is disposed outside the inner cover 3 and the stent body 2 is covered with the outer cover 4 (step SC3).

Next, the joining portions 5 are formed by joining the inner cover 3 and the outer cover 4 with each other in the regions of the protrusions 21b (step SC4).

Next, by removing the core rod 21 from inside the inner cover 3, the covered stent having the slack portions 6 is manufactured (step SC5).

Accordingly, with the manufacturing method of this embodiment, the slack portions 6 are formed by disposing the stent body 2 on the inner cover 3; therefore, special push tools 30 are not required. In addition, the entire inner cover 3 is uniformly pressed radially inward by the stent body 2; therefore, it is possible to form the slack portions 6 having desired dimensions by easily and accurately controlling the dimensions of the slack portions 6 even if the push tools 30 are not used.

In addition, only the inner cover 3 has the slack portions 6 and the outer cover 4 does not have slack portions; therefore, it is possible to decrease the sliding resistance exerted when the covered stent is released from the delivery system.

In this embodiment, the plurality of depressions 21a in the circumferential direction and the longitudinal direction are continuous with each other; however, alternatively, the depressions 21a in the two directions may be independent of each other, as in the depressions 20a in the first embodiment.

### Fourth Embodiment

Next, a covered stent manufacturing method and a covered stent according to a fourth embodiment of the present invention will be described.

In this embodiment, configurations that are different from those of the first embodiment will be described and configurations that are the same as those of the first embodiment will be given the same reference signs, and the descriptions thereof will be omitted.

The covered stent according to this embodiment includes the stent body 2, the inner cover 3, and the outer cover 4. The covered stent of this embodiment differs from the covered stent 1 of the first embodiment in that the inner cover 3 does not have slack portions 6 and the outer cover 4 has the slack portions 7 that expand radially outward.

The outer cover 4 has the plurality of slack portions 7 that are arrayed in the longitudinal direction with spacings therebetween. Each of the slack portions 7 consists of a depression that extends over the entire circumference of the outer cover 4 and expands radially outward. Some of the engagement portions 2b are disposed between the inner cover 3 and the slack portions 7. The slack portions 7 form spaces between the inner cover 3 and the outer cover 4, and, in addition, create slack in the outer cover 4. Therefore, as in the inner cover 3 of the first embodiment, the outer cover 4 allows movements of the pair of bent portions 2c, 2d, forming each of the engagement portions 2b, in the longitudinal direction and the radial direction, and thus, the covers 3, 4 are prevented from hindering the three-dimensional displacements of the bent portions 2c, 2d when the covered stent is bent. Accordingly, a low axial force and high flexibility are realized in the covered stent.

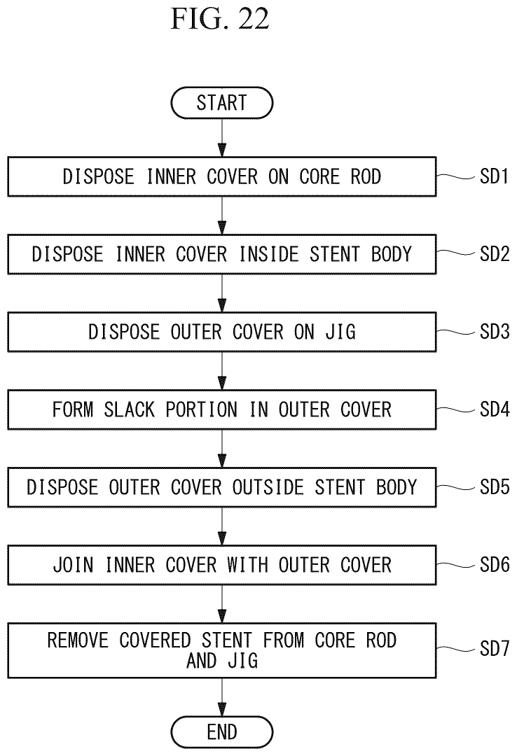

As shown in FIG. 22, the covered stent manufacturing method according to this embodiment includes: step SD1 of disposing the inner cover 3 on a first jig 22; step SD2 of disposing the inner cover 3 inside the stent body 2; step SD3 of disposing the outer cover 4 on a second jig 23; step SD4 of forming the slack portions 7 in the outer cover 4; step SD5 of disposing the outer cover 4 outside the inner cover 3; step SD6 of joining the inner cover 3 and the outer cover 4 with each other; and step SD7 of removing the covered stent from the jigs 22 and 23.

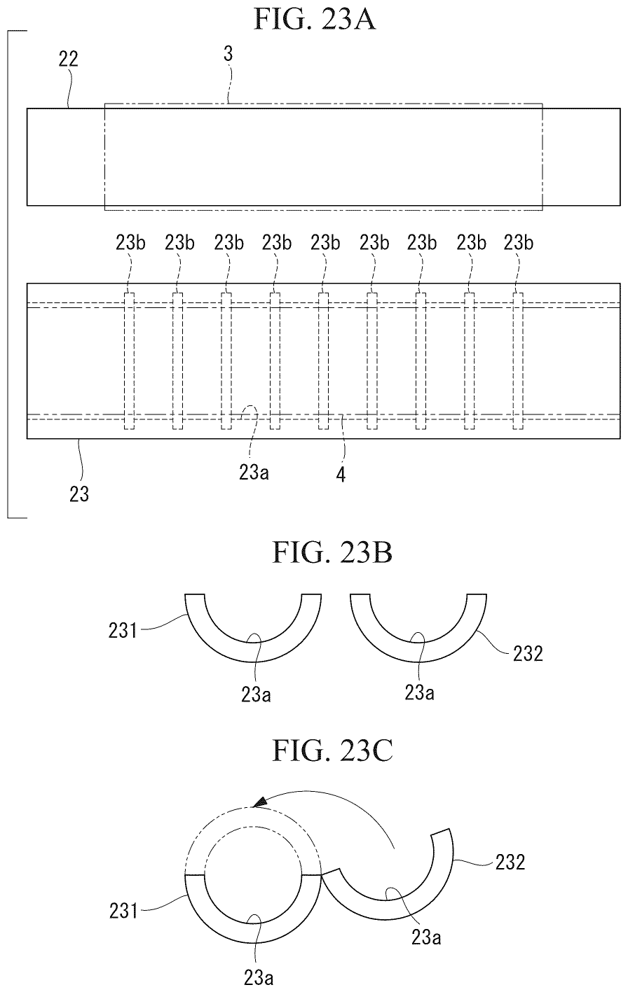

FIGS. 23A to 23C show the jigs 22 and 23 used in this embodiment.

The first jig 22 is a columnar core rod and an outer surface of the core rod 22 is a cylindrical surface without unevenness.

The second jig 23 is a cylindrical member and, on a cylindrical inner surface (mounting surface) 23a of the second jig 23, a plurality of depressions 23b that are arrayed in the longitudinal direction with spacings therebetween are formed. The plurality of depressions 23b are structures for forming the slack portions 7. Each of the depressions 23b extends over the entire circumference and are depressed radially outward. As shown in FIGS. 23B and 23C, in order to make it possible to expose the inner surface 23a, the second jig 23 may consist of two half bodies 231 and 232 that are semi-cylindrical. The two half bodies 231 and 232 may be separated from each other (see FIG. 23B) or may be connected so as to allow opening/closing thereof (see FIG. 23C).

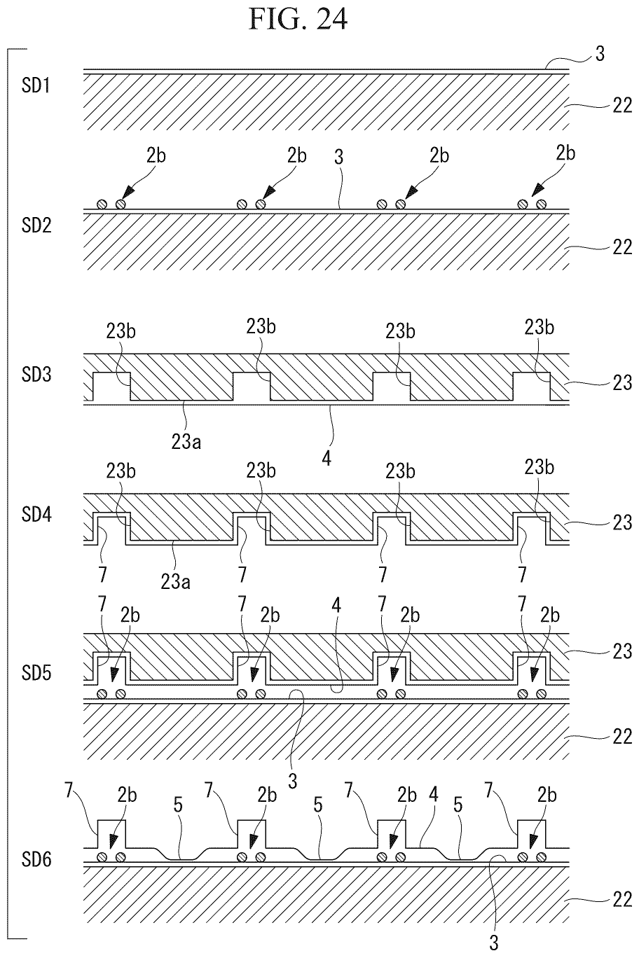

As shown in FIG. 24, the inner cover 3 is disposed on the outer surface of the core rod 22 (step SD1), as in step SA1. Next, the stent body 2 is disposed on the outer surface of the inner cover 3 (step SD2), as in step SA3.

Next, the outer cover 4 is disposed on the inner surface 23a of the second jig 23 (step SD3).

Next, the slack portions 7 are formed by pushing the outer cover 4 into the depressions 23b by using the push tools 30 (not shown) (step SD4).

Next, the outer cover 4 is disposed outside the stent body 2 by inserting the core rod 22 into the second jig 23 (step SD5). Here, the outer cover 4 is aligned with respect to the stent body 2 at a position at which a joining mechanism (not shown) provided in the second jig 23 is disposed at a center of the mesh of the stent body 2. The joining mechanism is for the joining of the covers 3, 4. For example, the joining mechanism is a pin that protrudes from the inner surface 23a of the second jig 23 and pressurizes or heats the covers 3, 4 or is a through-hole through which a separate joining tool passes from outside to inside the second jig 23.

Next, the covers 3, 4 are joined with each other in some regions on the inner side of the mesh by utilizing the joining mechanism, and the joining portions 5 are consequently formed (step SD6).

Next, by removing the core rod 22 from inside the inner cover 3, the covered stent having the slack portions 7 is manufactured (step SD7).

Accordingly, with the manufacturing method of this embodiment, it is possible to manufacture the covered stent with a low axial force and high flexibility, in which the outer cover 4 has slack due to the slack portions 7 and the covers 3, 4 do not hinder bending of the stent body 2.

In addition, the slack portions 7 having the dimensions equivalent to those of the depressions 23b are formed by pushing the outer cover 4 into the depressions 23b of the second jig 23. Accordingly, it is possible to form the slack portions 7 having desired dimensions by easily and accurately controlling the dimensions of the slack portions 7.

In addition, of the covers 3, 4 forming a double structure, only the outer cover 4 has the slack portions 7 and the inner cover 3 does not have slack portions. Therefore, the inner surface of the covered stent is a smooth surface without unevenness and a substance in the body can smoothly flow in the hollow portion inside the covered stent.

In this embodiment, the inner cover 3 does not have slack portions; however, alternatively, the inner cover 3 may have the slack portions 6 described in the first to third embodiments.

In this case, instead of the first jig 22, a first jig having depressions, such as the core rod 20, 21 of the first to third embodiments, is used in combination with the second jig 23. Accordingly, it is possible to manufacture the covered stent in which the inner cover 3 has the radial slack portions 6 that expand radially inward and the outer cover 4 has the slack portions 7 that expand radially outward. Furthermore, it is possible to vary the dimensions and the shapes of the slack portions 6 and the slack portions 7 with respect to each other.

### Fifth Embodiment

Next, a covered stent manufacturing method and a covered stent according to a fifth embodiment of the present invention will be described.

This embodiment is a modification of the fourth embodiment and differs from the fourth embodiment in terms of the covered stent manufacturing method. In this embodiment, configurations that are different from those of the first and fourth embodiments will be described and configurations that are the same as those of the first and fourth embodiments will be given the same reference signs, and the descriptions thereof will be omitted.

The covered stent according to this embodiment includes the stent body 2, the inner cover 3, and the outer cover 4, and the outer cover 4 has the slack portions 7 that expand radially outward.

The covered stent manufacturing method according to this embodiment includes steps SD1, SD2, SD3, SD4, SD5, SD6, and SD7 described in the fourth embodiment.

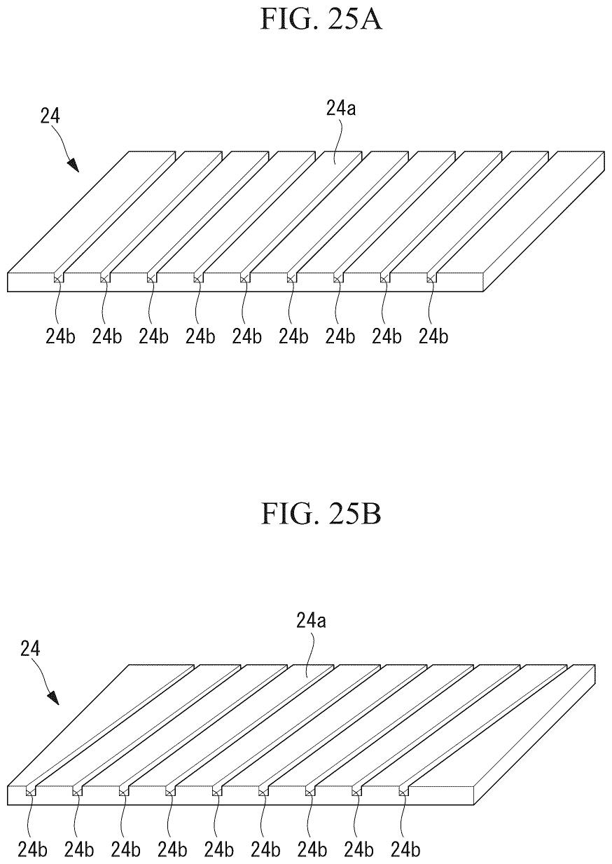

FIG. 25A shows a second jig 24 used in this embodiment. The second jig 24 is a flat member having a rectangular flat mounting surface 24a and a plurality of depressions 24b that are arrayed in the longitudinal direction with spacings therebetween are formed on the mounting surface 24a. The depressions 24b are structures for forming the slack portions 7. Each of the depressions 24b consists of a groove that extends over the entire width of the mounting surface 24a. The second jig 24 may be formed from a hard material or may be formed from a flexible material, for example, silicone.

In this embodiment, the outer cover 4 is disposed on the mounting surface 24a of the second jig 24 (step SD3).

Next, the slack portions 7 are formed by pushing the outer cover 4 into the depressions 24b by using the push tools 30 (step SD4).

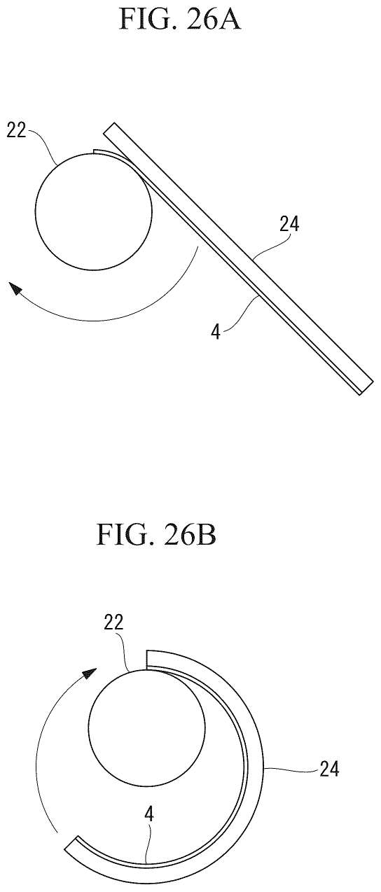

Next, as shown in FIG. 26A, the outer cover 4 is disposed on the stent body 2 by winding the outer cover 4 around the core rod 21 by rotating the mounting surface 24a about the core rod 22 (step SD5). After the outer cover 4 is disposed, the second jig 24 is removed. In FIGS. 26A and 26B, the illustrations of the stent body 2 and the inner cover 3 are omitted.

In the case in which the second jig 24 possesses flexibility, as shown in FIG. 26B, the outer cover 4 may be disposed on the stent body 2 by winding the second jig 24 around the core rod 22. In this case, the inner cover 3 and the outer cover 4 may be joined with each other (step SD6) by using the joining mechanism or the like described in the fourth embodiment while the state in which the second jig 24 is wound around the core rod 22 is maintained, and the second jig 24 may subsequently be removed.

Accordingly, with the manufacturing method of this embodiment, the second jig 24 has a simple shape in which the depressions 24b are machined on the flat mounting surface 24a. Therefore, it is possible to form the depressions 24b having various shapes and dimensions in a highly precise manner and it is possible to manufacture a covered stent having the slack portions 7 having various shapes and dimensions.

FIG. 25B shows another example of the second jig 24. Accordingly, it is possible to easily form the depressions 24b in an inclined direction. The second jig 24 in FIG. 26B is used in combination with the stent body 2 that is formed by knitting the wires 2a while winding the wires in a spiraling manner and in which the engagement portions 2b are arrayed in a spiraling manner.

In this embodiment, the covered stent in which the inner cover 3 has the slack portions 6 and the outer cover 4 has the slack portions 7 may be manufactured, as in the fourth embodiment, by using the first jig having the depressions and the second jig 24 in combination.

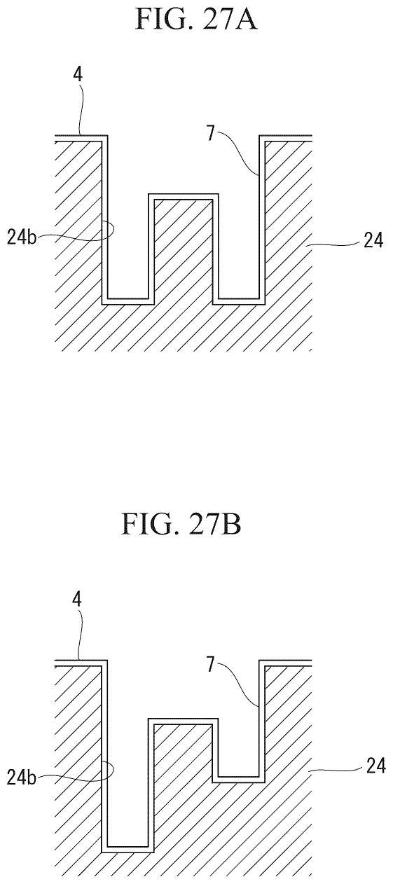

In this embodiment, the slack portions 6, 7 may have the shapes that deform into the prescribed folded shapes shown in FIGS. 8A and 9A, as in the first embodiment. FIGS. 27A and 27B show other examples of the shapes of the slack portions 6, 7 that are deformed into the prescribed folded shapes due to the compression in the radial direction. Accordingly, the shapes of the slack portions 6, 7 can be changed in various manners.

In the above-described first, second, fourth, and fifth embodiments, the push tools 30 are used as means for pushing the covers 3, 4 into the depressions 20a, 23b, 24b; however, alternatively, other means may be used.

For example, suction holes may be provided in inner surfaces of the depressions 20a, 23b, 24b and the covers 3, 4 may be sucked into the depressions 20a, 23b, 24b by means of suction. By employing such a means also, it is possible to push the covers 3, 4 into the depressions 20a, 23b, 24b and to cause deformations thereof along the inner surfaces of the depressions 20a, 23b, 24b.

### Sixth Embodiment

Next, a covered stent manufacturing method and a covered stent according to a sixth embodiment of the present invention will be described.

In this embodiment, configurations that are different from those of the first embodiment will be described and configurations that are the same as those of the first embodiment will be given the same reference signs, and the descriptions thereof will be omitted.

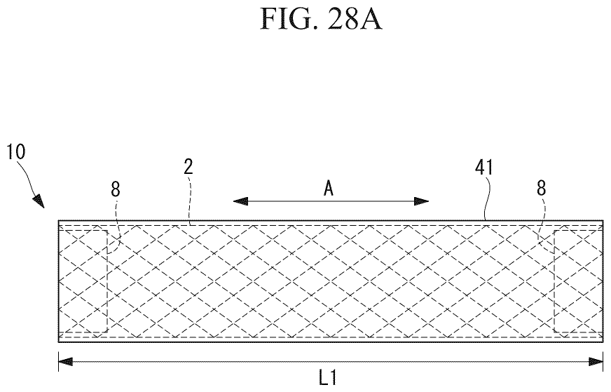

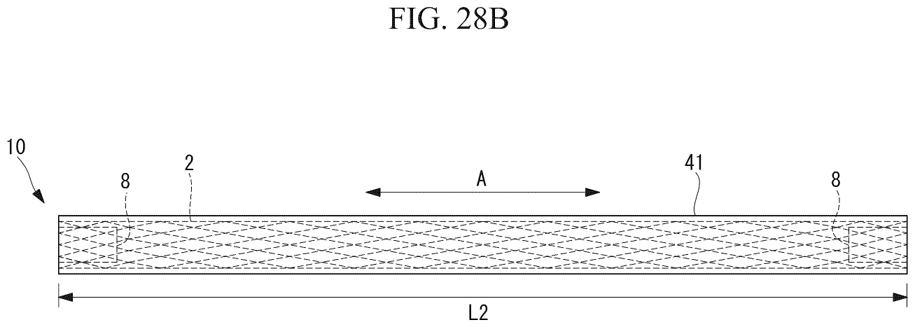

As shown in FIG. 28A, a covered stent 10 according to this embodiment includes: the stent body 2; a tubular outer cover 41 that covers the outside of the stent body 2; and securing covers 8 that are disposed inside the stent body 2 at two end portions thereof.

The stent body 2 can be deformed into a contracted state from an expanded state due to contraction in the radial direction and is mounted on a delivery system in the contracted state. As shown in FIG. 28B, the stent body 2 stretches in the longitudinal direction due to the contraction in the radial direction and a length L2 of the stent body 2 in the contracted state increases by, for example, about 20% to 50%, as compared with a length L1 of the stent body 2 in the expanded state.

The securing covers 8 are formed from an ePTFE and are disposed inside the stent body 2 over the entire circumference at the two end portions thereof. Two end portions of the outer cover 41 are joined with the securing covers 8 and the outer cover 41 is consequently connected to the stent body 2 at the two end portions.

The outer cover 41 is formed from an ePTFE and includes a stretching direction A in which high ductility is exhibited. Specifically, the outer cover 41 easily stretches in the stretching direction A and does not easily stretch in a direction orthogonal to the stretching direction. The stretching properties of such an outer cover 41 result from an ePTFE manufacturing method. The ePTFE manufacturing method includes a step of stretching PTFE and a step of sintering the stretched PTFE. Stretching the PTFE forms nodes that are distributed in an island-like manner and fibrils that extend in the stretching direction between the nodes, and the ePTFE exhibits high ductility in the stretching direction A in which the fibrils are oriented.

The outer cover 41 is disposed in the orientation in which the stretching direction A is aligned with the longitudinal direction of the stent body 2. In addition, in the expanded state, the outer cover 41 has slack in the longitudinal direction. In other words, the total length of the outer cover 41 corresponds to the length in which an extra length is added to the length L1 of the stent body 2 in the expanded state. Such an outer cover 41 is freely deformed in accordance with an external force in the expanded state and allows movements of the pair of bent portions 2c, 2d, forming each of the engagement portions 2b, in the longitudinal direction and the radial direction. Therefore, the outer cover 41 is prevented from hindering the three-dimensional displacements of the bent portions 2c, 2d when the covered stent 10 is bent and a low axial force and high flexibility are realized in the covered stent 10.

Furthermore, the stretching direction A of the outer cover 41 is aligned with the stretching direction of the stent body 2 when being contracted; therefore, the outer cover 41 is prevented from being broken or peeled off from the stent body 2 due to the stretching of the stent body 2. In the case in which the ductility of the outer cover is low in the stretching direction of the stent body, breakage, such as the outer cover being broken or being peeled off from the stent body, could occur due to the contraction of the covered stent.

Next, the covered stent manufacturing method according to this embodiment will be described.

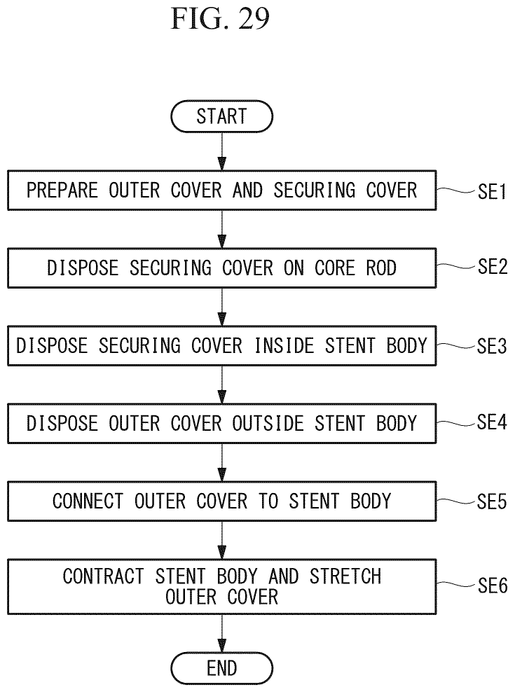

As shown in FIG. 29, the covered stent manufacturing method includes: step SE1 of preparing the outer cover 41 and the securing covers 8; step SE2 of disposing the securing covers 8 on the jig 22; step SE3 of disposing the securing covers 8 inside the stent body 2; step SE4 of disposing the outer cover 41 outside the stent body 2; step SE5 of partially connecting the outer cover 41 with the stent body 2; and step SE6 of stretching the outer cover 41 by contracting the stent body 2 in the radial direction.

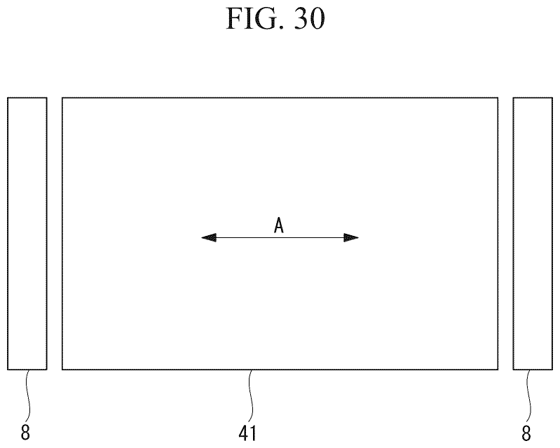

As shown in FIG. 30, the rectangular outer cover 41 and the band-like securing covers 8 out cut out from ePTFE sheets in consideration of the stretching direction A of the ePTFE (step SE1). The outer cover 41 includes a length direction and a width direction that respectively correspond to the longitudinal direction and the circumferential direction of the stent body 2. The outer cover 41 is cut out from the sheet so that the length direction of the outer cover 41 is aligned with the stretching direction A of the ePTFE.

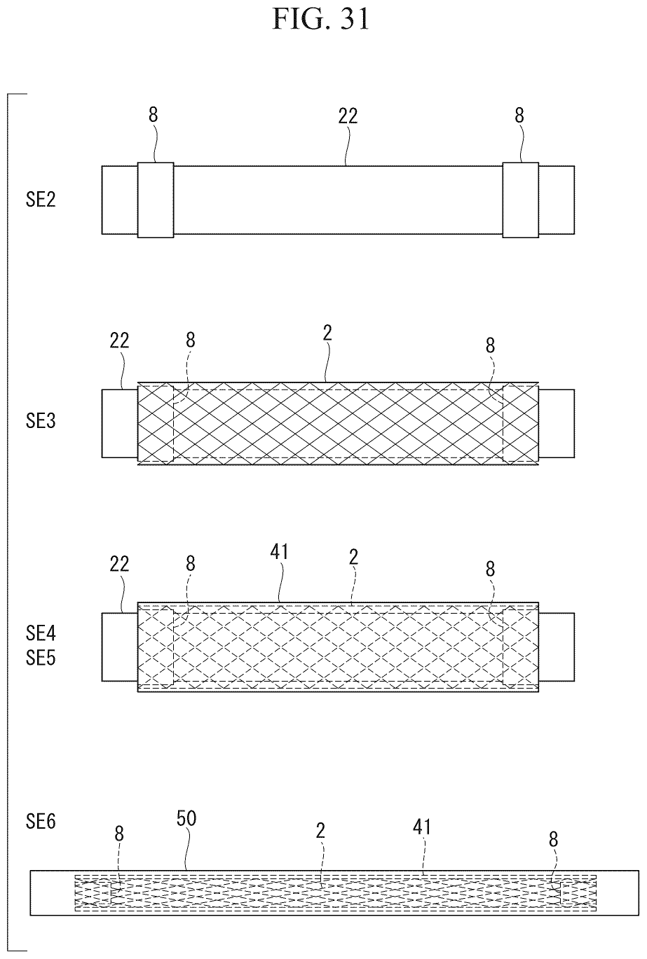

Next, as shown in FIG. 31, the securing covers 8 are wound around two sites in the jig 22 (step SE2). The jig 22 is a core rod having a cylindrical outer surface without unevenness.

Next, the securing covers 8 are disposed inside the stent body 2 at the two end portions thereof by inserting the core rod 22 into the stent body 2 (step SE3).

Next, the outer cover 41 is disposed outside the stent body 2 by aligning the length direction of the outer cover 41 with the longitudinal direction of the stent body 2 and winding the outer cover 41 around the core rod 22 over the entire circumference thereof (step SE4).

Next, the outer cover 41 is connected to the stent body 2 by joining the two end portions of the outer cover 41 with the securing covers 8 by means of an arbitrary joining method, such as thermocompression bonding or an adhesive (step SE5). In step SE5, the outer cover 41 is formed into a tubular shape by also joining end portions of the outer cover 41 in the width direction with other portions of the outer cover 41 over the entire length thereof. By removing the core rod 22 from inside the stent body 2 after the joining, an assembly of the stent body 2, the outer cover 41, and the securing covers 8 is removed from the core rod 22.

Next, the outer cover 41 is stretched in the longitudinal direction together with the stent body 2 by contracting the stent body 2 in the radial direction (step SE6). Step SE6 may be performed by inserting the assembly into a tube 50 having an inner diameter that is smaller than the diameter of the assembly in the expanded state. Accordingly, the length of the outer cover 41 increases and slack is formed in the outer cover 41 with respect to the length of the stent body 2 in the expanded state.

Accordingly, with the manufacturing method of this embodiment, slack for allowing the three-dimensional displacements of the bent portions 2c, 2d when the stent body 2 is bent is formed in the outer cover 41 simply by contracting the stent body 2, to which the outer cover 41 is partially connected, in the radial direction. Accordingly, it is possible to easily manufacture the covered stent 10 with a low axial force and high flexibility.

Furthermore, the stretching direction A of the outer cover 41 is aligned with the stretching direction of the stent body 2 when being contracted; therefore, it is possible to easily manufacture the covered stent 10 in which breakage is less likely to occur and that is highly reliable.

In this embodiment, the outer cover 41 is connected to the stent body 2 at the two end portions thereof; however, the number and the positions of connecting portions of the outer cover 41 with respect to the stent body 2 can be changed, as appropriate. For example, the outer cover 41 may be connected to the stent body 2 at a center portion in addition to the two end portions in order to increase the securing force of the outer cover 41 with respect to the stent body 2.

In the connecting portions joined by means of thermocompression bonding or the like, the ductility of the ePTFE deteriorates because the node and fibril structures are broken. Therefore, the number of the connecting portions is preferably small.

In this embodiment, the outer cover 41 is stretched by inserting the assembly into the tube 50; however, alternatively, the outer cover 41 may be stretched by mounting the assembly on a delivery system.

The delivery system has a tubular sheath to be inserted into a body cavity and the covered stent 10 is mounted on a distal-end portion of the sheath. After step SE5, by inserting the assembly into the sheath, it is possible to stretch the outer cover 41 by contracting the stent body 2 at the same time as the mounting on the delivery system.

In this embodiment, the manufacturing method includes step SE6 of stretching the outer cover 41; however, step SE6 may not be included. In this case, the covered stent 10 is provided in a state in which the outer cover 41 is not stretched.

The unstretched outer cover 41 is stretched by, for example, a user himself/herself mounting the covered stent 10 on the delivery system. Accordingly, it is possible to form slack in the outer cover 41 and realize a low axial force in the covered stent 10.

### Seventh Embodiment

Next, a covered stent manufacturing method and a covered stent according to a seventh embodiment of the present invention will be described.

In this embodiment, configurations that are different from those of the first and sixth embodiments will be described and configurations that are the same as those of the first and sixth embodiments will be given the same reference signs, and the descriptions thereof will be omitted.

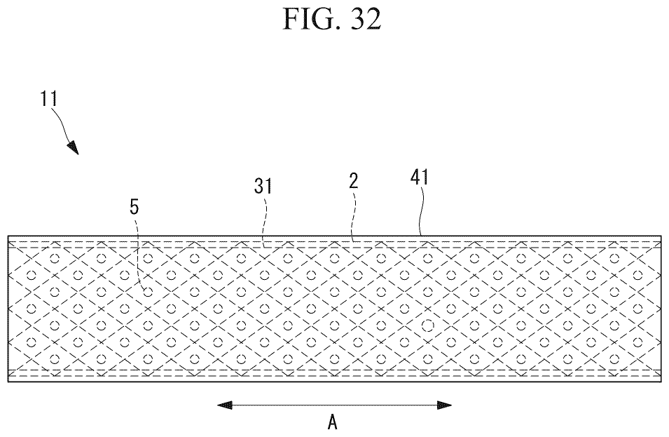

As shown in FIG. 32, a covered stent 11 according to this embodiment differs from the covered stent 10 of the sixth embodiment in that the covered stent 11 additionally includes a tubular inner cover 31 that covers the inside of the stent body 2 in addition to the stent body 2 and the outer cover 41.

The covers 31, 41 are partially joined with each other and are consequently partially connected to the stent body 2. For example, the covers 31, 41 are joined with each other in the joining portions 5, which are some regions on the inner side of the mesh of the stent body 2, and are separated from each other in the portions other than the joining portions 5.

The inner cover 31 is formed from an ePTFE, includes the stretching direction A in which high ductility is exhibited, and is disposed in the orientation in which the stretching direction A is aligned with the longitudinal direction of the stent body 2, as in the outer cover 41. In addition, in the expanded state, the inner cover 31 has slack in the longitudinal direction. In other words, the total length of the inner cover 31 corresponds to the length in which an extra length is added to the length L1 of the stent body 2 in the expanded state.

Such an inner cover 31 allows the movements of the pair of bent portions 2c, 2d, forming each of the engagement portions 2b, in the longitudinal direction and the radial direction, as in the outer cover 41. Therefore, the covers 31, 41 are prevented from hindering the three-dimensional displacements of the bent portions 2c, 2d when the covered stent 11 is bent, and a low axial force and high flexibility are realized in the covered stent 11.

Furthermore, the stretching directions A of the covers 31, 41 are aligned with the stretching direction of the stent body 2 when being contracted; therefore, the covers 31, 41 are prevented from being broken or being peeled off from the stent body 2 due to the stretching of the stent body 2.

Next, the covered stent manufacturing method according to this embodiment will be described.

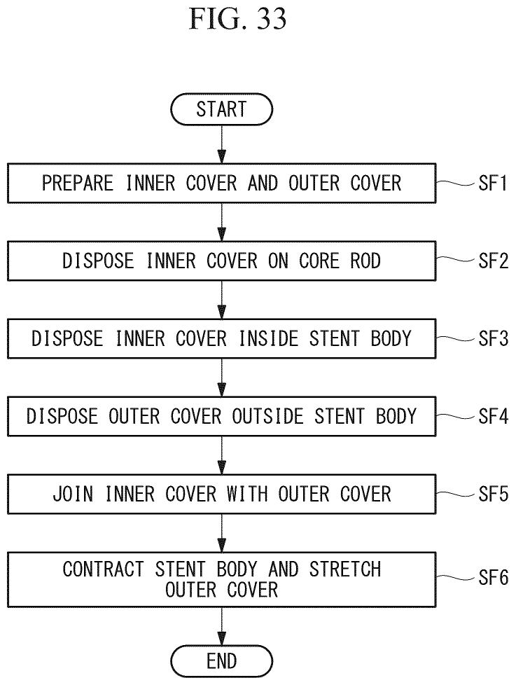

As shown in FIG. 33, the covered stent manufacturing method includes: step SF1 of preparing the inner cover 31 and the outer cover 41; step SF2 of disposing the inner cover 31 on the jig 22; step SF3 of disposing the inner cover 31 inside the stent body 2; step SF4 of disposing the outer cover 41 outside the stent body 2; step SF5 of partially connecting the inner cover 31 and the outer cover 41 to the stent body 2; and step SF6 of stretching the inner cover 31 and the outer cover 41 by contracting the stent body 2 in the radial direction.

As in step SE1, the rectangular outer cover 41 and the rectangular inner cover 31 are cut out from ePTFE sheets in consideration of the stretching direction A of the ePTFE so that the length directions of the covers 31, 41 are aligned with the stretching direction A (step SF1).

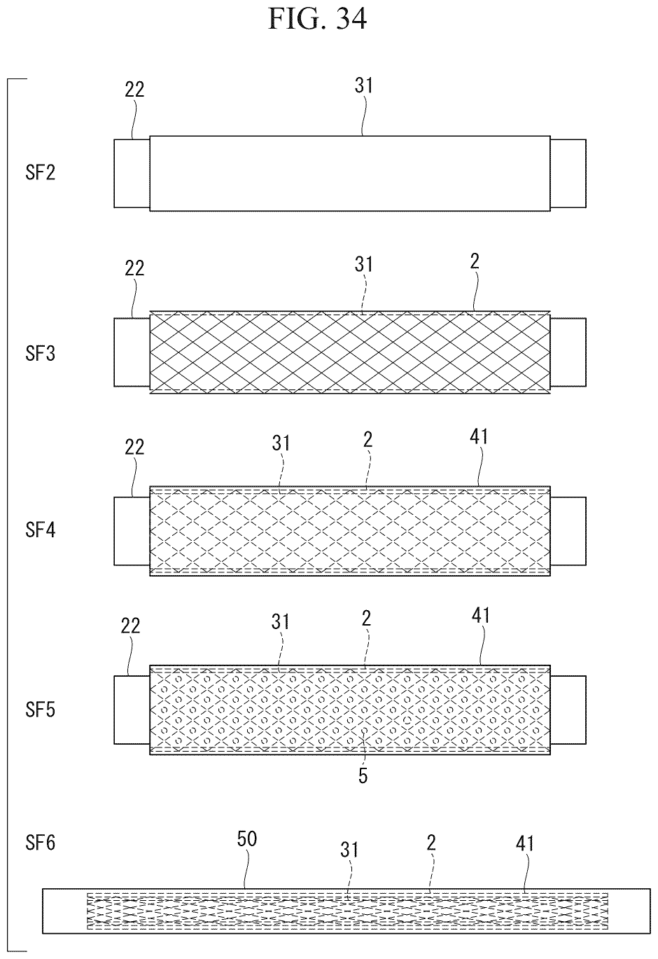

Next, as shown in FIG. 34, the inner cover 31 is disposed on the outer surface of the core rod 22 by aligning the length direction of the inner cover 31 with the longitudinal direction of the core rod 22 and winding the inner cover 31 around the core rod 22 over the entire circumference thereof (step SF2). In step SF2, the inner cover 31 is formed into a tubular shape by joining end portions of the inner cover 31 in the width direction with other portions of the inner cover 31 over the entire length thereof.

Next, the stent body 2 is disposed outside the inner cover 31 by inserting the core rod 22 into the stent body 2 (step SF3).

Next, the outer cover 41 is disposed outside the stent body 2 by aligning the length direction of the outer cover 41 with the longitudinal direction of the stent body 2 and winding the outer cover 41 around the core rod 22 over the entire circumference thereof (step SF4). In step SF4, the outer cover 41 is formed into a tubular shape by joining end portions of the outer cover 41 in the width direction with other portions of the outer cover 41 over the entire length thereof.

Next, the covers 31, 41 are connected to the stent body 2 by forming the joining portions 5 by joining the outer cover 41 and the inner cover 31 with each other by means of an arbitrary joining method, such as thermocompression bonding or an adhesive, in some regions on the inner side of the mesh (step SF5). By removing the core rod 22 from the inner cover 31 after the joining, an assembly of the stent body 2 and the covers 31, 41 is removed from the core rod 22.

Next, as in step SE6, the covers 31, 41 are stretched in the longitudinal direction together with the stent body 2 by contracting the stent body 2 in the radial direction (step SF6). Accordingly, the length of each of the covers 31, 41 increases and slack is formed in each of the covers 31, 41 with respect to the length of the stent body 2 in the expanded state.

Accordingly, with the manufacturing method of this embodiment, slack for allowing the three-dimensional displacements of the bent portions 2c, 2d when the stent body 2 is bent is simultaneously formed in the covers 31, 41 simply by contracting the stent body 2, to which the covers 31, 41 forming the double structure are partially connected, in the radial direction. Accordingly, it is possible to easily manufacture the covered stent 11 with a low axial force and high flexibility. Furthermore, the stretching directions A of the covers 31, 41 are aligned with the stretching direction of the stent body 2 when being contracted; therefore, it is possible to easily manufacture the covered stent 11 in which breakage of the covers 31, 41 is less likely to occur and that is highly reliable.

In step SF6 of this embodiment, as in the sixth embodiment, the covers 31, 41 may be stretched by mounting the assembly on the delivery system instead of the tube 50.

In this embodiment, as in the sixth embodiment, the manufacturing method may not include step SF6 and the covered stent 11 may be provided in a state in which the covers 31, 41 are not stretched.

In the sixth and seventh embodiments, the stretching directions A of the covers 31, 41 are aligned with the longitudinal direction of the stent body 2; however, in the case in which the wires 2a are wound around the stent body 2 in a spiraling manner, the stretching direction A may be aligned with the stretching directions of the wires 2a when the stent body 2 is contracted.

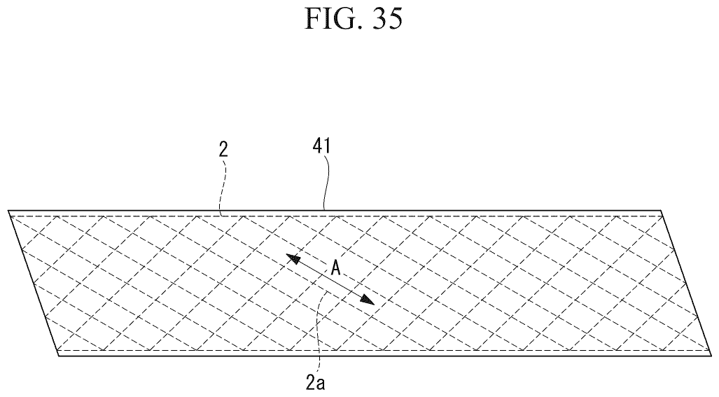

As shown in FIG. 35, the stent body 2 formed by knitting the wires 2a while winding the wires in a spiraling manner stretches when the diameter thereof decreases in the radial direction, while being rotated in a direction in which twist thereof is released. Therefore, as a result of aligning the stretching direction A with the stretching directions of the wires 2a, it is possible to effectively stretch the covers 31, 41. In this case, in step SE1, SF1, the covers 31, 41 are cut out from ePTFE sheets so that the length directions of the covers 31, 41 are inclined with respect to the stretching direction A by an angle according to the angle of the wires 2a in the stretching directions.

In the sixth and seventh embodiments, the stent body 2 is not limited to a stent body having the engagement portions 2b, and a stent body having other structures in which the stent body stretches in the longitudinal direction due to the contraction in the radial direction may be employed. In addition, although examples in which the covers 31, 41 are disposed over the entire length of the stent body 2 have been described, the same effects are also afforded in the case in which the covers are disposed in a portion of the stent body 2.

Furthermore, in the first to fifth embodiments also, the covers made of the ePTFE and the stent body can be disposed so that the stretching directions of the covers 31, 41 are aligned with the stretching direction of the stent body 2.

As above, although the embodiments and modifications of the present invention have been described in detail with reference to the drawings, the specific configurations are not limited to the above-described embodiments and design alterations, etc. within the range that does not depart from the scope of the present invention are also encompassed. In addition, the constituent elements indicated in the above-described embodiments and modifications can be configured in combination, as appropriate.

### REFERENCE SIGNS LIST

1, 10, 11 covered stent
2 stent body
2a wire
2b engagement portion
2c, 2d bent portion
3, 31 inner cover
4, 41 outer cover
5 joining portion
6, 7 slack portion
20, 21, 22 first jig (core rod)
23, 24 second jig
23a, 24a mounting surface
20a, 21a, 23b, 24b depression

1. A covered stent manufacturing method comprising:
disposing an inner cover inside a stent body having a mesh structure formed by knitting wires, the stent body having engagement portions, each of which is formed by hooking two bent portions of the wires with each other;
disposing an outer cover outside the stent body;
forming a slack portion that expands in a radial direction of the stent body in at least one of the inner cover or the outer cover, the slack portion creating slack that allows movements of the two bent portions in a longitudinal direction and the radial direction of the stent body in at least one of the inner cover or the outer cover; and
joining the inner cover and the outer cover with each other in regions on an inner side of the mesh of the mesh structure.

2. The covered stent manufacturing method according to claim 1, further comprising disposing the inner cover on an outer surface of a columnar first jig, wherein forming the slack portion includes forming the slack portion in the inner cover by pushing the inner cover into depressions formed on the outer surface of the first jig before disposing the outer cover.

3. The covered stent manufacturing method according to claim 2, wherein forming the slack portion includes forming the slack portion in the outer cover by pushing the outer cover into the depressions of the first jig after disposing the outer cover.

4. The covered stent manufacturing method according to claim 1, further comprising disposing the inner cover on an outer surface of a columnar first jig, wherein forming the slack portion includes forming the slack portion in each of the inner cover and the outer cover by simultaneously pushing the inner cover and the outer cover into depressions formed on the outer surface of the first jig after disposing the outer cover.

5. The covered stent manufacturing method according to claim 4, wherein forming the slack portion and joining are simultaneously performed by joining, while the inner cover and the outer cover are pushed into the depressions by using a joining tool, the inner cover and the outer cover with each other by means of the joining tool.

6. The covered stent manufacturing method according to claim 1, further comprising disposing the inner cover on an outer surface of a columnar first jig, wherein disposing the stent body is disposing the stent body on an outer surface of the inner cover by aligning protrusions provided on the outer surface of the first jig with the regions on the inner side of the mesh of the mesh structure and the slack portion is consequently formed in the inner cover.

7. The covered stent manufacturing method according to claim 1, further comprising disposing the outer cover on a mounting surface of a second jig, wherein forming the slack portion includes forming the slack portion in the outer cover by pushing the outer cover into depressions formed on the mounting surface of the second jig before disposing the outer cover outside the stent body.

8. The covered stent manufacturing method according to claim 7, wherein the second jig has a cylindrical member, an inner surface of which serves as the mounting surface, or a flat member having the mounting surface that is flat.

9. The covered stent manufacturing method according to claim 1, wherein the slack portion extends in a circumferential direction of the stent body.

10. The covered stent manufacturing method according to claim 9, wherein the inner cover and the outer cover are joined with each other in a region between the two engagement portions adjacent to each other disposed in the slack portion.

11. The covered stent manufacturing method according to claim 9, wherein:
a plurality of slack portions that are arrayed in the longitudinal direction of the stent body with spacings between each other are formed; and
the inner cover and the outer cover are joined with each other in a region between the two slack portions adjacent to each other.

12. The covered stent manufacturing method according to claim 1, wherein:
the slack portion has a shape that deforms into a prescribed folded shape due to a compression force in the radial direction; and,
in the prescribed folded shape, a portion of the cover constituting the slack portion is stacked in the radial direction outside an opening of the slack portion.

13. The covered stent manufacturing method according to claim 1, wherein:
the inner cover is made of an ePTFE; and
the inner cover is disposed with respect to the stent body in an orientation in which a stretching direction of the ePTFE is aligned with a stretching direction of the stent body when the stent body is contracted in the radial direction.

14. The covered stent manufacturing method according to claim 1, wherein:
the outer cover is made of an ePTFE; and
the outer body is disposed with respect to the stent body in an orientation in which a stretching direction of the ePTFE is aligned with a stretching direction of the stent body when the stent body is contracted in the radial direction.

15. A covered stent comprising:
a stent body that has a mesh structure formed by knitting wires and that has engagement portions, each of which is formed by hooking two bent portions of the wires with each other;
an inner cover that covers an inside of the stent body; and
an outer cover that covers an outside of the stent body, wherein
at least one of the inner cover or the outer cover has a slack portion that expands in a radial direction of the stent body, the slack portion creating slack that allows movements of the two bent portions in a longitudinal direction and the radial direction of the stent body in at least one of the inner cover or the outer cover, and
the inner cover and the outer cover are joined with each other in regions on an inner side of the mesh of the mesh structure.

16. The covered stent according to claim 15, wherein:
the inner cover is made of an ePTFE; and
the inner cover is disposed with respect to the stent body in an orientation in which a stretching direction of the ePTFE is aligned with a stretching direction of the stent body when the stent body is contracted in the radial direction.

17. The covered stent according to claim 15, wherein:
the outer cover is made of an ePTFE; and
the outer body is disposed with respect to the stent body in an orientation in which a stretching direction of the ePTFE is aligned with a stretching direction of the stent body when the stent body is contracted in the radial direction.

18. A covered stent manufacturing method comprising:
disposing, at least inside or outside a stent body, a cover made of an ePTFE in an orientation in which a stretching direction of the ePTFE is aligned with a stretching direction of the stent body when the stent body is contracted in a radial direction;
partially connecting the cover with the stent body; and
creating slack in the cover by contracting the stent body in the radial direction and stretching the cover.

19. The covered stent manufacturing method according to claim 18, wherein contracting the stent body in the radial direction and stretching the cover are performed in a step of mounting the stent body, to which the cover is connected, on a delivery system.
